(19) United States
(12) Patent Application Publication
(10) Pub. No.: US 2024/0423539 A1
(43) Pub. Date: Dec. 26, 2024

(54) System for Measuring Thoracic Fluid Using Radiofrequency

(71) Applicant: ZOLL Medical Israel Ltd., Kfar-Saba (IL)

(72) Inventors: Bahar Davoudi, Pittsburgh, PA (US); Rafael Ravid, Savyon (IL); Uriel Weinstein, Mazkeret Batya (IL); Ramu Perumal, Gibsonia, PA (US); Kent J. Volosin, Big Sky, MT (US)

(21) Appl. No.: 18/684,390

(22) PCT Filed: Aug. 15, 2022
(86) PCT No.: PCT/IL2022/050884

§ 371 Date: Feb. 16, 2024

Related U.S. Application Data
(60) Provisional application No. 63/260,410, filed on Aug. 19, 2021.

(51) Int. Cl. A61B 5/00; A61B 5/05; A61B 5/11; A61B 5/282; A61B 5/318; G16H 10/60; G16H 40/67

(52) U.S. Cl. CPC A61B 5/4875; A61B 5/05; A61B 5/1118; A61B 5/282; A61B 5/318; A61B 5/6805; A61B 5/7475; G16H 10/60; G16H 40/67; A61B 5/6833; A61B 2562/0219

(57) ABSTRACT

A patient monitoring system for improved thoracic fluid measurement from a patient is provided. The system includes therefrom. The system also includes a remote server including a memory and a processor configured to receive the RF value(s), retrieve weighting parameter(s), and apply the weighting parameter(s) to the RF value(s) to determine and output an amount of thoracic fluid. The weighting parameter(s) is determined by a clinical modality process including receiving reference thoracic fluid information for a patient population, producing RF-based thoracic fluid information for the patient population, and deriving the weighting parameter(s) by optimizing an association of the RF-based thoracic fluid information with the reference thoracic fluid information.

### CROSS-REFERENCE TO RELATED APPLICATIONS

This application claims priority to U.S. Provisional Patent Application Ser. No. 63/260,410, filed on Aug. 19, 2021, titled “SYSTEM FOR MEASURING THORACIC FLUID USING RADIOFREQUENCY,” the entirety of which is hereby incorporated by reference.

### BACKGROUND

The present disclosure relates to directing radiofrequency (RF) waves into a patient and using the waves to determine, measure, and/or monitor thoracic fluid of the patient.

There is a wide variety of electronic and mechanical devices for monitoring patients' underlying medical conditions. In some examples, depending on the underlying medical condition being monitored and/or treated, medical devices such as pressure sensing devices, cardiac pacemakers, or defibrillators may be surgically implanted or connected externally to the patient. Physicians may use such devices alone or in combination with drug therapies to treat or control patients' medical conditions.

Such patients can include heart failure patients. For example, congestive heart failure (CHF) is a condition in which the heart's function as a pump is inadequate to meet the body's needs. Generally, many disease processes can impair the pumping efficiency of the heart to cause congestive heart failure. The symptoms of congestive heart failure vary, but can include fatigue, diminished exercise capacity, shortness of breath, and swelling (edema). The diagnosis of congestive heart failure is based on knowledge of the individual's medical history, a careful physical examination, and selected laboratory tests.

Heart failure patients can also benefit from having their thoracic fluid levels monitored. RF electromagnetic radiation has been used for diagnosis and imaging of body tissues. Diagnostic devices that include an antenna can be used to direct the RF electromagnetic waves into a body and generate signals responsively to the waves that are transmitted through and/or scattered from within the body. Such signals can be processed to determine various properties of body tissues located along the paths of the transmitted and/or scattered waves.

### SUMMARY

In one or more examples, a patient monitoring system for thoracic fluid measurement (e.g., improved thoracic fluid measurement) from a patient is provided. The system includes an ambulatory medical device configured to be worn by the patient for an extended period of time. The ambulatory medical device includes at least one RF antenna and associated RF circuitry configured to transmit RF waves in a range from about 0.1 GHz to about 5 GHz towards a thoracic region of the patient and receive RF waves reflected from the thoracic region and/or transmitted through the thoracic region to generate at least one RF value therefrom. The system also includes a remote server in communication with the ambulatory medical device. The remote server includes a memory implemented in non-transitory media and a processor in communication with the memory. The processor is configured to receive the at least one RF value for the patient and retrieve at least one weighting parameter from the memory. The at least one weighting parameter is determined by a computer-tomography (CT) process including receiving reference CT scan-based thoracic fluid information for a patient population, producing RF-based thoracic fluid information for the patient population, and deriving the at least one weighting parameter by optimizing an association of the RF-based thoracic fluid information with the reference CT scan-based thoracic fluid information for the patient population. The processor is configured to apply the at least one weighting parameter to the at least one RF value to determine an amount of thoracic fluid in the patient and output the determined amount of thoracic fluid in the patient. The terms “includes” and “comprises” may be used interchangeably herein.

Implementations of the patient monitoring system for improved thoracic fluid measurement from a patient can include one or more of the following optional features. The ambulatory medical device includes a plurality of ECG electrodes configured to sense ECG signals of the patient. The ambulatory medical device includes a motion sensor configured to acquire motion signals associated with the patient. The ambulatory medical device includes a garment configured to be worn about a torso of the patient. The ambulatory medical device includes an adhesive patch configured to be adhered to skin of the patient and a monitoring unit configured to be removably attached to the adhesive patch. The monitoring unit includes the at least one RF antenna and associated RF circuitry.

The CT process includes identifying patient health information for the patient population and deriving the at least one weighting parameter by further optimizing an association of the RF-based thoracic fluid information and the patient health information with the reference CT scan-based thoracic fluid information for the patient population. The processor is further configured to identify at least one patient health value for the patient. Applying the at least one weighting parameter includes applying the at least one weighting parameter to the at least one RF value and to the at least one patient health value to determine the amount of thoracic fluid in the patient. The patient health information for the patient population includes demographic information for the patient population. The at least one patient health value for the patient includes at least one demographic of the patient. The patient health information for the patient population includes biometric information for the patient population. The at least one patient health value for the patient includes at least one biometric of the patient. The patient health information the patient population includes anthropomorphic information for the patient population. The at least one patient health value for the patient includes at least one anthropomorphic parameter of the patient. The patient health information for the patient population includes at least one of gender information, age information, height information, weight information, body mass index (BMI) information, heart rate information, respiration rate information, heart failure type information, comorbidity information, cardiac events information, chest circumference information, or thoracic thickness information for the patient population. The at least one patient health value for the patient includes at least one of gender, age, height, weight, BMI, a heart rate, a respiration rate, a heart failure type, a comorbidity, a cardiac event, a chest circumference, or a thoracic tissue thickness of the patient. Identifying the at least one patient health value for the patient includes receiving the at least one patient health value as a user input. The ambulatory medical device includes a plurality of ECG electrodes configured to sense ECG signals of the patient. The processor is further configured to receive the ECG signals, and identifying the at least one patient health value for the patient includes determining a heart rate for the patient from the ECG signals. The ambulatory medical device includes a motion sensor configured to acquire motion signals associated with the patient. The processor is further configured to receiving the motion signals, and identifying the at least one patient health value for the patient includes determining a respiration rate for the patient from the motion signals.

Producing the RF-based thoracic fluid information for the patient population includes receiving RF value information for the patient population and, for each individual of the patient population, indexing the individual's RF value information by normalizing the RF value information to a baseline. Applying the at least one weighting parameter to the at least one RF value includes indexing the at least one RF value by normalizing the at least one RF value to the baseline. Producing the RF-based thoracic fluid information for the patient population includes receiving RF value information for the patient population and, for each individual of the patient population, transforming the individual's RF value information using an alpha function. The alpha function includes an exponential function based on a rate of attenuation in the received RF waves reflected from the thoracic region and/or transmitted through the thoracic region compared to the RF waves transmitted from the at least one RF antenna. Applying the at least one weighting parameter to the at least one RF value includes transforming the at least one RF value using the alpha function. Producing the RF-based thoracic fluid information for the patient population includes receiving RF value information for the patient population and, for each individual of the patient population, transforming the individual's RF value information using at least one of a linear function, an nth-order polynomial function, a logarithmic function, an exponential function, or a power function. Applying the at least one weighting parameter to the at least one RF value includes transforming the at least one RF value using at least one of a linear function, an nth-order polynomial function, a logarithmic function, an exponential function, or a power function.

The reference CT scan-based thoracic fluid information for the patient population includes reference CT scan-based extravascular lung fluid information for the patient population. The determined amount of thoracic fluid in the patient includes a determined amount of extravascular lung fluid in the patient. The reference CT scan-based thoracic fluid information for the patient population includes reference CT scan-based extravascular and intravascular lung fluid information for the patient population. The determined amount of thoracic fluid in the patient includes a determined amount of extravascular and intravascular lung fluid in the patient.

The processor is further configured to identify at least one calibration value for the patient. Applying the at least one weighting parameter to the at least one RF value to determine the amount of thoracic fluid in the patient includes applying the at least one weighting parameter to the at least one RF value and to the at least one calibration value to determine the amount of thoracic fluid in the patient. Applying the at least one weighting parameter to the at least one RF value to determine the amount of thoracic fluid in the patient includes applying the at least one weighting parameter to the at least one RF value to produce a preliminary amount of thoracic fluid in the patient and adjusting the preliminary amount of thoracic fluid in the patient by the at least one calibration value to produce the amount of thoracic fluid in the patient. Identifying the at least one calibration value for the patient includes receiving at least one CT scan value for the patient. The at least one CT scan value for the patient is based on a reference CT scan for the patient. Identifying the at least one calibration value for the patient includes receiving at least one of a BMI, a chest circumference, or a thoracic tissue thickness for the patient. Identifying the at least one calibration value for the patient includes determining a rate of attenuation in the received RF waves reflected from the thoracic region and/or transmitted through the thoracic region compared to the RF waves transmitted from the at least one RF antenna and determining the at least one calibration value for the patient based on the rate of attenuation in the reflected RF waves and/or transmitted RF waves.

Deriving the at least one weighting parameter includes deriving the at least one weighting parameter by optimizing the association of the RF-based thoracic fluid information with the reference CT scan-based thoracic fluid information for the patient population using machine learning. Optimizing the association of the RF-based thoracic fluid information with the reference CT scan-based thoracic fluid information for the patient population includes optimizing a correlation of the RF-based thoracic fluid information with the reference CT scan-based thoracic fluid information.

In one or more examples, a patient monitoring system for thoracic fluid measurement (e.g., improved thoracic fluid measurement) from a patient is provided. The system includes an ambulatory medical device configured to be worn by the patient for an extended period of time. The ambulatory medical device includes at least one RF antenna and associated RF circuitry configured to transmit RF waves in a range from about 0.1 GHz to about 5 GHz towards a thoracic region of the patient and receive RF waves reflected from the thoracic region and/or transmit through the thoracic region to generate at least one RF value therefrom. The ambulatory medical device also includes a memory implemented in a non-transitory media and a processor in communication with the memory. The processor is configured to retrieve at least one weighting parameter from the memory. The at least one weighting parameter is determined by a CT process including receiving reference CT scan-based thoracic fluid information for a patient population, producing RF-based thoracic fluid information for the patient population, and deriving the at least one weighting parameter by optimizing an association of the RF-based thoracic fluid information with the reference CT scan-based thoracic fluid information for the patient population. The processor is further configured to apply the at least one weighting parameter to the at least one RF value to determine an amount of thoracic fluid in the patient and output the determined amount of thoracic fluid in the patient.

Implementations of this patient monitoring system for improved thoracic fluid measurement from a patient can include one or more of the features discussed with respect to the patient monitoring system for improved thoracic fluid measurement above. Implementations of this patient monitoring system for improved thoracic fluid measurement from a patient can additionally and/or alternatively include one or more of the following features. The processor is further configured to transmit the determined amount of thoracic fluid to a remote server in communication with the ambulatory medical device. The CT process further includes identifying patient health information for the patient population and deriving the at least one weighting parameter by further optimizing an association of the RF-based thoracic fluid information and the patient health information with the reference CT scan-based thoracic fluid information for the patient population. The processor is further configured to identify at least one patient health value for the patient. Applying the at least one weighting parameter includes applying the at least one weighting parameter to the at least one RF value and to the at least one patient health value to determine the amount of thoracic fluid in the patient. The ambulatory medical device includes a plurality of ECG electrodes configured to sense ECG signals of the patient. Identifying the at least one patient health value for the patient includes determining a heart rate for the patient from the ECG signals. The ambulatory medical device includes a motion sensor configured to acquire motion signals associated with the patient. Identifying the at least one patient health value for the patient includes determining a respiration rate for the patient from the motion signals.

In one or more examples, a patient monitoring system for thoracic fluid measurement (e.g., improved thoracic fluid measurement) from a patient includes an ambulatory medical device configured to be worn by the patient for an extended period of time. The ambulatory medical device includes at least one RF antenna and associated RF circuitry configured to transmit RF waves in a range from about 0.1 GHz to about 5 GHz towards a thoracic region of the patient and receive RF waves reflected from the thoracic region and/or transmitted through the thoracic region to generate at least one RF value therefrom. The system also includes a remote server in communication with the ambulatory medical device. The remote server includes a memory implemented in a non-transitory media and a processor in communication with the memory. The processor is configured to receive the at least one RF value for the patient and retrieve at least one weighting parameter from the memory. The at least one weighting parameter is determined by a clinical modality process including receiving reference thoracic fluid information for a patient population, producing RF-based thoracic fluid information for the patient population, and deriving the at least one weighting parameter by optimizing an association of the RF-based thoracic fluid information with the reference thoracic fluid information for the patient population. The processor is further configured to apply the at least one weighting parameter to the at least one RF value to determine an amount of thoracic fluid in the patient and output the determined amount of thoracic fluid in the patient.

Implementations of this patient monitoring system for improved thoracic fluid measurement from a patient can include one or more of the features discussed with respect to the patient monitoring systems for improved thoracic fluid measurement above. Implementations of this patient monitoring system for improved thoracic fluid measurement from a patient can additionally and/or alternatively include one or more of the following features. The clinical modality process further includes identifying patient health information for the patient population and deriving the at least one weighting parameter by further optimizing an association of the RF-based thoracic fluid information and the patient health information with the reference thoracic fluid information for the patient population. The processor is further configured to identify at least one patient health value for the patient. Applying the at least one weighting parameter includes applying the at least one weighting parameter to the at least one RF value and to the at least one patient health value to determine the amount of thoracic fluid in the patient.

The reference thoracic fluid information for the patient population includes reference extravascular lung fluid information for the patient population. The determined amount of thoracic fluid in the patient includes a determined amount of extravascular lung fluid in the patient. The reference thoracic fluid information for the patient population includes reference extravascular and intravascular lung fluid information for the patient population. The determined amount of thoracic fluid in the patient includes a determined amount of extravascular and intravascular lung fluid in the patient.

The processor is further configured to identify at least one calibration value for the patient. Identifying the at least one calibration value for the patient includes receiving at least clinical modality value for the patient. The at least one clinical modality value for the patient is based on a reference clinical modality scan, image, or process for the patient.

Deriving the at least one weighting parameter includes deriving the at least one weighting parameter by optimizing the association of the RF-based thoracic fluid information with the reference thoracic fluid information for the patient population using machine learning. Optimizing the association of the RF-based thoracic fluid information with the reference thoracic fluid information for the patient population includes optimizing a correlation of the RF-based thoracic fluid information with the reference thoracic fluid information.

In one or more examples, a method for thoracic fluid measurement (e.g., improved thoracic fluid measurement) from a patient is performed. In implementations, the method includes transmitting, by at least one RF antenna and associated circuitry, RF waves in a range from about 0.1 GHz to about 5 GHz towards a thoracic region of the patient. The method includes receiving, by the at least one RF antenna and associated circuitry, RF waves reflected from the thoracic region and/or transmitted through the thoracic region to generate at least one RF value therefrom; and retrieving at least one weighting parameter from a memory. The at least one weighting parameter is determined by a CT process including receiving reference CT scan-based thoracic fluid information for a patient population, producing RF-based thoracic fluid information for the patient population, and deriving the at least one weighting parameter by optimizing an association of the RF-based thoracic fluid information with the reference CT scan-based thoracic fluid information for the patient population. The method also includes applying the at least one weighting parameter to the at least one RF value to determine an amount of thoracic fluid in the patient and outputting the determined amount of thoracic fluid in the patient.

Implementations of the method for improved thoracic fluid measurement from a patient can include one or more of the following optional features. The method includes transmitting the at least one RF value to a remote server. The remote server includes the memory. The method includes storing the at least one RF value in the memory. The method includes transmitting the determined amount of thoracic fluid to a remote server. The method includes sensing, by a plurality of ECG electrodes, ECG signals of the patient. The method includes acquiring, by a motion sensor, motion signals associated with the patient.

The CT process further includes identifying patient health information for the patient population and deriving the at least one weighting parameter by further optimizing an association of the RF-based thoracic fluid information and the patient health information with the reference CT scan-based thoracic fluid information for the patient population. The method includes identifying at least one patient health value for the patient. Applying the at least one weighting parameter further includes applying the at least one weighting parameter to the at least one RF value and to the at least one patient health value to determine the amount of thoracic fluid in the patient. The patient health information for the patient population includes demographic information for the patient population. The at least one patient health value for the patient includes at least one demographic of the patient. The patient health information for the patient population includes biometric information for the patient population. The at least one patient health value for the patient includes at least one biometric of the patient. The patient health information the patient population includes anthropomorphic information for the patient population. The at least one patient health value for the patient includes at least one anthropomorphic parameter of the patient. The patient health information for the patient population includes at least one of gender information, age information, height information, weight information, body mass index (BMI) information, heart rate information, respiration rate information, heart failure type information, comorbidity information, cardiac events information, chest circumference information, or thoracic thickness information for the patient population. The at least one patient health value for the patient includes at least one of gender, age, height, weight, BMI, a heart rate, a respiration rate, a heart failure type, a comorbidity, a cardiac event, a chest circumference, or a thoracic tissue thickness of the patient. Identifying the at least one patient health value for the patient includes receiving the at least one patient health value as a user input. The method includes sensing, by a plurality of ECG electrodes, ECG signals of the patient. The method includes receiving the ECG signals, and identifying the at least one patient health value for the patient includes determining a heart rate for the patient from the ECG signals. The method includes acquiring, by a motion sensor, motion signals associated with the patient. The method includes receiving the motion signals, and identifying the at least one patient health value for the patient includes determining a respiration rate for the patient from the motion signals.

Producing the RF-based thoracic fluid information for the patient population includes receiving RF value information for the patient population and, for each individual of the patient population, indexing the individual's RF value information by normalizing the RF value information to a baseline. Applying the at least one weighting parameter to the at least one RF value includes indexing the at least one RF value by normalizing the at least one RF value to the baseline. Producing the RF-based thoracic fluid information for the patient population includes receiving RF value information for the patient population and, for each individual of the patient population, transforming the individual's RF value information using an alpha function. The alpha function includes an exponential function based on a rate of attenuation in the received RF waves reflected from the thoracic region and/or transmitted through the thoracic region compared to the RF waves transmitted by the at least one RF antenna and associated circuitry. Applying the at least one weighting parameter to the at least one RF value includes transforming the at least one RF value using the alpha function. Producing the RF-based thoracic fluid information for the patient population includes receiving RF value information for the patient population and, for each individual of the patient population, transforming the individual's RF value information using at least one of a linear function, an nth-order polynomial function, a logarithmic function, an exponential function, or a power function. Applying the at least one weighting parameter to the at least one RF value includes transforming the at least one RF value using at least one of a linear function, an nth-order polynomial function, a logarithmic function, an exponential function, or a power function.

The reference CT scan-based thoracic fluid information for the patient population includes reference CT scan-based extravascular lung fluid information for the patient population. The determined amount of thoracic fluid in the patient includes a determined amount of extravascular lung fluid in the patient. The reference CT scan-based thoracic fluid information for the patient population includes reference CT scan-based extravascular and intravascular lung fluid information for the patient population. The determined amount of thoracic fluid in the patient includes a determined amount of extravascular and intravascular lung fluid in the patient.

The method includes identifying at least one calibration value for the patient. Applying the at least one weighting parameter to the at least one RF value to determine the amount of thoracic fluid in the patient includes applying the at least one weighting parameter to the at least one RF value and to the at least one calibration value to determine the amount of thoracic fluid in the patient. Applying the at least one weighting parameter to the at least one RF value to determine the amount of thoracic fluid in the patient includes applying the at least one weighting parameter to the at least one RF value to produce a preliminary amount of thoracic fluid in the patient and adjusting the preliminary amount of thoracic fluid in the patient by the at least one calibration value to produce the amount of thoracic fluid in the patient. Identifying the at least one calibration value for the patient includes receiving at least one CT scan value for the patient. The at least one CT scan value for the patient is based on a reference CT scan for the patient. Identifying the at least one calibration value for the patient includes receiving at least one of a BMI, a chest circumference, or a thoracic tissue thickness for the patient. Identifying the at least one calibration value for the patient includes determining a rate of attenuation in the received RF waves reflected from the thoracic region and/or transmitted through the thoracic region compared to the RF waves transmitted from the at least one RF antenna and determining the at least one calibration value for the patient based on the rate of attenuation in the reflected RF waves and/or transmitted RF waves.

Deriving the at least one weighting parameter includes deriving the at least one weighting parameter by optimizing the association of the RF-based thoracic fluid information with the reference CT scan-based thoracic fluid information for the patient population using machine learning. Optimizing the association of the RF-based thoracic fluid information with the reference CT scan-based thoracic fluid information for the patient population includes optimizing a correlation of the RF-based thoracic fluid information with the reference CT scan-based thoracic fluid information.

In one or more examples, a method for thoracic fluid measurement (e.g., improved thoracic fluid measurement) from a patient is provided. In implementations, the method includes transmitting, by at least one RF antenna and associated circuitry, RF waves in a range from about 0.1 GHz to about 5 GHz towards a thoracic region of the patient. The method includes receiving, by the at least one RF antenna and associated circuitry, RF waves reflected from the thoracic region and/or transmitted through the thoracic region to generate at least one RF value therefrom; and retrieving at least one weighting parameter from a memory. The at least one weighting parameter is determined by a clinical modality process including receiving reference thoracic fluid information for a patient population, producing RF-based thoracic fluid information for the patient population, and deriving the at least one weighting parameter by optimizing an association of the RF-based thoracic fluid information with the reference thoracic fluid information for the patient population. The method also includes applying the at least one weighting parameter to the at least one RF value to determine an amount of thoracic fluid in the patient, and outputting the determined amount of thoracic fluid in the patient.

Implementations of this method for improved thoracic fluid measurement from a patient can include one or more of the features discussed with respect to the method for improved thoracic fluid measurement from a patient above. Implementations of this method for improved thoracic fluid measurement from a patient can additionally and/or alternatively include one or more of the following features. The clinical modality process further includes identifying patient health information for the patient population and deriving the at least one weighting parameter by further optimizing an association of the RF-based thoracic fluid information and the patient health information with the reference thoracic fluid information for the patient population. The method includes identifying at least one patient health value for the patient. Applying the at least one weighting parameter further includes applying the at least one weighting parameter to the at least one RF value and to the at least one patient health value to determine the amount of thoracic fluid in the patient.

The reference thoracic fluid information for the patient population includes reference extravascular lung fluid information for the patient population. The determined amount of thoracic fluid in the patient includes a determined amount of extravascular lung fluid in the patient. The reference thoracic fluid information for the patient population includes extravascular and intravascular lung fluid for the patient population. The determined amount of thoracic fluid in the patient includes a determined amount of extravascular and intravascular lung fluid in the patient.

The method includes identifying at least one calibration value for the patient. The at least one clinical modality value for the patient is based on a reference clinical modality scan, image, or process for the patient.

Deriving the at least one weighting parameter includes deriving the at least one weighting parameter by optimizing the association of the RF-based thoracic fluid information with the reference thoracic fluid information for the patient population using machine learning. Optimizing the association of the RF-based thoracic fluid information with the reference thoracic fluid information for the patient population includes optimizing a correlation of the RF-based thoracic fluid information with the reference thoracic fluid information.

In one or more examples, a non-transitory computer-readable medium storing sequences of instructions executable by at least one processor is provided. The sequences of instructions instruct the at least one processor to perform thoracic fluid measurements (e.g., improved thoracic improvement measurements, or improved thoracic fluid measurements) from a patient. The sequences of instructions include instructions to receive at least one RF value for the patient from an ambulatory medical device configured to be worn by the patient for an extended period of time (e.g., from at least one RF antenna and associated RF circuitry) and retrieve at least one weighting parameter from a memory. The at least one weighting parameter is determined by a CT process including receiving reference CT scan-based thoracic fluid information for a patient population, producing RF-based thoracic fluid information for the patient population, and deriving the at least one weighting parameter by optimizing an association of the RF-based thoracic fluid information with the reference CT scan-based thoracic fluid information for the patient population. The sequences of instructions also include instructions to apply the at least one weighting parameter to the at least one RF value to determine an amount of thoracic fluid in the patient and output the determined amount of thoracic fluid in the patient.

Implementations of the non-transitory computer-readable medium storing sequences of instructions executable by at least one processor can include one or more of the following features. The sequences of instructions include instructions to receive ECG signals from the ambulatory medical device. The sequences of instructions include instructions to receive motion signals associated with the patient from the ambulatory medical device. The CT process further includes identifying patient health information for the patient population and deriving the at least one weighting parameter by further optimizing an association of the RF-based thoracic fluid information and the patient health information with the reference CT scan-based thoracic fluid information for the patient population. The sequences of instructions further include instructions to identify at least one patient health value for the patient. The instructions to apply the at least one weighting parameter further include instructions to apply the at least one weighting parameter to the at least one RF value and to the at least one patient health value to determine the amount of thoracic fluid in the patient. The patient health information for the patient population includes demographic information for the patient population. The at least one patient health value for the patient includes at least one demographic of the patient. The patient health information for the patient population includes biometric information for the patient population. The at least one patient health value for the patient includes at least one biometric of the patient. The patient health information the patient population includes anthropomorphic information for the patient population. The at least one patient health value for the patient includes at least one anthropomorphic parameter of the patient. The patient health information for the patient population includes at least one of gender information, age information, height information, weight information, body mass index (BMI) information, heart rate information, respiration rate information, heart failure type information, comorbidity information, cardiac events information, chest circumference information, or thoracic thickness information for the patient population. The at least one patient health value for the patient includes at least one of gender, age, height, weight, BMI, a heart rate, a respiration rate, a heart failure type, a comorbidity, a cardiac event, a chest circumference, or a thoracic tissue thickness of the patient. The instructions to identify the at least one patient health value for the patient include instructions to receive the at least one patient health value as a user input. The sequences of instructions include instructions to receive ECG signals from the ambulatory medical device, and the instructions to identify the at least one patient health value for the patient include instructions to determine a heart rate for the patient from the ECG signals. The sequences of instructions include instructions to receive motion signals from the ambulatory medical device, and the instructions to identify the at least one patient health value for the patient include instructions to determine a respiration rate for the patient from the motion signals.

Producing the RF-based thoracic fluid information for the patient population includes receiving RF value information for the patient population and, for each individual of the patient population, indexing the individual's RF value information by normalizing the RF value information to a baseline. The instructions to apply the at least one weighting parameter to the at least one RF value include instructions to index the at least one RF value by normalizing the at least one RF value to the baseline. Producing the RF-based thoracic fluid information for the patient population includes receiving RF value information for the patient population and, for each individual of the patient population, transforming the individual's RF value information using an alpha function. The alpha function includes an exponential function based on a rate of attenuation in RF waves reflected from a thoracic region of the patient and/or transmitted through the thoracic region compared to RF waves transmitted by the ambulatory medical device into the thoracic region, wherein the RF waves reflected from the thoracic region and/or transmitted through the thoracic region are received at the ambulatory medical device and used to produce the at least one RF value. The instructions to apply the at least one weighting parameter to the at least one RF value include instructions to transform the at least one RF value using the alpha function. Producing the RF-based thoracic fluid information for the patient population include receiving RF value information for the patient population and, for each individual of the patient population, transform the individual's RF value information using at least one of a linear function, an nth-order polynomial function, a logarithmic function, an exponential function, or a power function. The instructions to apply the at least one weighting parameter to the at least one RF value include instructions to transform the at least one RF value using at least one of a linear function, an nth-order polynomial function, a logarithmic function, an exponential function, or a power function.

The reference CT scan-based thoracic fluid information for the patient population includes reference CT scan-based extravascular lung fluid information for the patient population. The determined amount of thoracic fluid in the patient includes a determined amount of extravascular lung fluid in the patient. The reference CT scan-based thoracic fluid information for the patient population includes reference CT scan-based extravascular and intravascular lung fluid information for the patient population. The determined amount of thoracic fluid in the patient includes a determined amount of extravascular and intravascular lung fluid in the patient.

The sequences of instructions further include instructions to identify at least one calibration value for the patient. The instructions to apply the at least one weighting parameter to the at least one RF value to determine the amount of thoracic fluid in the patient include instructions to apply the at least one weighting parameter to the at least one RF value and to the at least one calibration value to determine the amount of thoracic fluid in the patient. The instructions to apply the at least one weighting parameter to the at least one RF value to determine the amount of thoracic fluid in the patient include instructions to applying the at least one weighting parameter to the at least one RF value to produce a preliminary amount of thoracic fluid in the patient and adjusting the preliminary amount of thoracic fluid in the patient by the at least one calibration value to produce the amount of thoracic fluid in the patient. The instructions to identify the at least one calibration value for the patient include instructions to receive at least one CT scan value for the patient. The at least one CT scan value for the patient is based on a reference CT scan for the patient. The instructions to identify the at least one calibration value for the patient include instructions to receive at least one of a BMI, a chest circumference, or a thoracic tissue thickness for the patient. The instructions to identify the at least one calibration value for the patient include instructions to determine a rate of attenuation in the received RF waves reflected from the thoracic region and/or transmitted through the thoracic region compared to the RF waves transmitted from the at least one RF antenna and determine the at least one calibration value for the patient based on the rate of attenuation in the reflected RF waves and/or transmitted RF waves.

Deriving the at least one weighting parameter includes deriving the at least one weighting parameter by optimizing the association of the RF-based thoracic fluid information with the reference CT scan-based thoracic fluid information for the patient population using machine learning. Optimizing the association of the RF-based thoracic fluid information with the reference CT scan-based thoracic fluid information for the patient population includes optimizing a correlation of the RF-based thoracic fluid information with the reference CT scan-based thoracic fluid information.

In one or more examples, a non-transitory computer-readable medium storing sequences of instructions executable by at least one processor is provided. The sequences of instructions instruct the at least one processor to perform thoracic fluid measurements (e.g., improved thoracic fluid measurements) from a patient. The sequences of instructions include instructions to receive at least one RF value for the patient from an ambulatory medical device configured to be worn by the patient for an extended period of time (e.g., from at least one RF antenna and associated circuitry) and retrieve at least one weighting parameter from a memory. The at least one weighting parameter is determined by a clinical modality process including receiving reference thoracic fluid information for a patient population, producing RF-based thoracic fluid information for the patient population, and deriving the at least one weighting parameter by optimizing an association of the RF-based thoracic fluid information with the reference thoracic fluid information for the patient population. The sequences of instructions further include instructions to apply the at least one weighting parameter to the at least one RF value to determine an amount of thoracic fluid in the patient and output the determined amount of thoracic fluid in the patient.

Implementations of the non-transitory computer-readable medium storing sequences of instructions executable by at least one processor can include one or more of the features discussed with respect to the non-transitory computer-readable medium above. Implementations of the non-transitory computer-readable medium storing sequences of instructions executable by at least one processor can additionally and/or alternatively include one or more of the following features. The clinical modality process further includes identifying patient health information for the patient population and deriving the at least one weighting parameter by further optimizing an association of the RF-based thoracic fluid information and the patient health information with the reference thoracic fluid information for the patient population. The sequences of instructions further include instructions to identify at least one patient health value for the patient. Applying the at least one weighting parameter further includes applying the at least one weighting parameter to the at least one RF value and to the at least one patient health value to determine the amount of thoracic fluid in the patient.

The reference thoracic fluid information for the patient population includes reference extravascular lung fluid information for the patient population. The determined amount of thoracic fluid in the patient includes a determined amount of extravascular lung fluid in the patient. The reference thoracic fluid information for the patient population includes reference extravascular and intravascular lung fluid information for the patient population. The determined amount of thoracic fluid in the patient includes a determined amount of extravascular and intravascular lung fluid in the patient.

The processor is further configured to identify at least one calibration value for the patient. The instructions to identify the at least one calibration value for the patient include instructions to receive at least clinical modality value for the patient. The at least one clinical modality value for the patient is based on a reference clinical modality scan, image, or process for the patient.

Deriving the at least one weighting parameter includes deriving the at least one weighting parameter by optimizing the association of the RF-based thoracic fluid information with the reference thoracic fluid information for the patient population using machine learning. Optimizing the association of the RF-based thoracic fluid information with the reference thoracic fluid information for the patient population includes optimizing a correlation of the RF-based thoracic fluid information with the reference thoracic fluid information.

In an aspect there is provided a patient monitoring system for improved thoracic fluid measurement from a patient, including an ambulatory medical device configured to be worn by the patient for an extended period of time, including at least one RF antenna and associated RF circuitry configured to transmit RF waves in a range from about 0.1 GHz to about 5 GHz towards a thoracic region of the patient and receive RF waves reflected from the thoracic region and/or transmitted through the thoracic region to generate at least one RF value therefrom; and a remote server in communication with the ambulatory medical device, the remote server including a memory implemented in a non-transitory media and; a processor in communication with the memory, the processor configured to receive the at least one RF value for the patient, retrieve at least one weighting parameter from the memory, the at least one weighting parameter determined by a computer-tomography (CT) process including receiving reference CT scan-based thoracic fluid information for a patient population; producing RF-based thoracic fluid information for the patient population; and deriving the at least one weighting parameter by optimizing an association of the RF-based thoracic fluid information with the reference CT scan-based thoracic fluid information for the patient population; apply the at least one weighting parameter to the at least one RF value to determine an amount of thoracic fluid in the patient, and output the determined amount of thoracic fluid in the patient.

In another aspect there is provided a patient monitoring system for improved thoracic fluid measurement from a patient, including an ambulatory medical device configured to be worn by the patient for an extended period of time, including at least one RF antenna and associated RF circuitry configured to transmit RF waves in a range from about 0.1 GHz to about 5 GHz towards a thoracic region of the patient and receive RF waves reflected from the thoracic region and/or transmit through the thoracic region to generate at least one RF value therefrom; a memory implemented in a non-transitory media; and a processor in communication with the memory, the processor configured to retrieve at least one weighting parameter from the memory, the at least one weighting parameter determined by a computer-tomography (CT) process including receiving reference CT scan-based thoracic fluid information for a patient population, producing RF-based thoracic fluid information for the patient population, and deriving the at least one weighting parameter by optimizing an association of the RF-based thoracic fluid information with the reference CT scan-based thoracic fluid information for the patient population, apply the at least one weighting parameter to the at least one RF value to determine an amount of thoracic fluid in the patient; and output the determined amount of thoracic fluid in the patient.

In another aspect there is provided a patient monitoring system for improved thoracic fluid measurement from a patient, including an ambulatory medical device configured to be worn by the patient for an extended period of time, including at least one RF antenna and associated RF circuitry configured to transmit RF waves in a range from about 0.1 GHz to about 5 GHz towards a thoracic region of the patient and receive RF waves reflected from the thoracic region and/or transmitted through the thoracic region to generate at least one RF value therefrom; and a remote server in communication with the ambulatory medical device, the remote server including a memory implemented in a non-transitory media; and a processor in communication with the memory, the processor configured to receive the at least one RF value for the patient, retrieve at least one weighting parameter from the memory, the at least one weighting parameter determined by a clinical modality process includes receiving reference thoracic fluid information for a patient population; producing RF-based thoracic fluid information for the patient population; and deriving the at least one weighting parameter by optimizing an association of the RF-based thoracic fluid information with the reference thoracic fluid information for the patient population; apply the at least one weighting parameter to the at least one RF value to determine an amount of thoracic fluid in the patient, and output the determined amount of thoracic fluid in the patient.

In another aspect there is provided a method for improved thoracic fluid measurement from a patient, including transmitting, by at least one RF antenna and associated circuitry, RF waves in a range from about 0.1 GHz to about 5 GHz towards a thoracic region of the patient; receiving, by the at least one RF antenna and associated circuitry, RF waves reflected from the thoracic region and/or transmitted through the thoracic region to generate at least one RF value therefrom; retrieving at least one weighting parameter from a memory, the at least one weighting parameter determined by a computer-tomography (CT) process including receiving reference CT scan-based thoracic fluid information for a patient population, producing RF-based thoracic fluid information for the patient population, and deriving the at least one weighting parameter by optimizing an association of the RF-based thoracic fluid information with the reference CT scan-based thoracic fluid information for the patient population, applying the at least one weighting parameter to the at least one RF value to determine an amount of thoracic fluid in the patient, and outputting the determined amount of thoracic fluid in the patient.

In another aspect there is provided a method for improved thoracic fluid measurement from a patient, including transmitting, by at least one RF antenna and associated circuitry, RF waves in a range from about 0.1 GHz to about 5 GHz towards a thoracic region of the patient; receiving, by the at least one RF antenna and associated circuitry, RF waves reflected from the thoracic region and/or transmitted through the thoracic region to generate at least one RF value therefrom; retrieving at least one weighting parameter from a memory, the at least one weighting parameter determined by a clinical modality process including receiving reference thoracic fluid information for a patient population, producing RF-based thoracic fluid information for the patient population, and deriving the at least one weighting parameter by optimizing an association of the RF-based thoracic fluid information with the reference thoracic fluid information for the patient population, applying the at least one weighting parameter to the at least one RF value to determine an amount of thoracic fluid in the patient, and outputting the determined amount of thoracic fluid in the patient.

In another aspect there is provided a non-transitory computer-readable medium storing sequences of instructions executable by at least one processor, the sequences of instructions instructing the at least one processor to perform improved thoracic fluid measurements from a patient, the sequences of instructions including instructions to receive at least one RF value for the patient from an ambulatory medical device configured to be worn by the patient for an extended period of time; retrieve at least one weighting parameter from a memory, the at least one weighting parameter determined by a computer-tomography (CT) process including receiving reference CT scan-based thoracic fluid information for a patient population, producing RF-based thoracic fluid information for the patient population, and deriving the at least one weighting parameter by optimizing an association of the RF-based thoracic fluid information with the reference CT scan-based thoracic fluid information for the patient population, apply the at least one weighting parameter to the at least one RF value to determine an amount of thoracic fluid in the patient; and output the determined amount of thoracic fluid in the patient.

In another aspect there is provided a non-transitory computer-readable medium storing sequences of instructions executable by at least one processor, the sequences of instructions instructing the at least one processor to perform improved thoracic fluid measurements from a patient, the sequences of instructions include instructions to receive at least one RF value for the patient from an ambulatory medical device configured to be worn by the patient for an extended period of time; retrieve at least one weighting parameter from a memory, the at least one weighting parameter determined by a clinical modality process including receiving reference thoracic fluid information for a patient population, producing RF-based thoracic fluid information for the patient population, and deriving the at least one weighting parameter by optimizing an association of the RF-based thoracic fluid information with the reference thoracic fluid information for the patient population, apply the at least one weighting parameter to the at least one RF value to determine an amount of thoracic fluid in the patient; and output the determined amount of thoracic fluid in the patient.

It will be appreciated that one or more of the optional features described with reference to one of the examples above or independent claims below may also be provided in combination with one or more of the aspects described above or independent claims below.

The claims are directed to a corresponding method, non-transitory computer-readable medium, and system. In an aspect, the independent method claim recites a method for thoracic fluid measurement from a patient, including receiving, by at least one RF antenna and associated circuitry of an ambulatory medical device configured to be worn by the patient for an extended period of time, RF waves reflected from the thoracic region of the patient and/or transmitted through the thoracic region to generate at least one RF value therefrom; retrieving at least one weighting parameter from a memory, the at least one weighting parameter determined by a clinical modality process comprising receiving reference clinical modality-based thoracic fluid information for a patient population, producing RF-based thoracic fluid information for the patient population, and deriving the at least one weighting parameter by optimizing an association of the RF-based thoracic fluid information with the reference clinical modality-based thoracic fluid information for the patient population, applying the at least one weighting parameter to the at least one RF value to determine an amount of thoracic fluid in the patient, and outputting the determined amount of thoracic fluid in the patient. In an aspect, the independent system claim and independent non-transitory computer-readable medium claim are directed to a respective system and non-transitory computer readable medium configured to perform the method. It will be appreciated that a feature described in relation to one category may be implemented as a corresponding feature in another category.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example patient monitoring system.

FIG. 2 depicts an example ambulatory medical device being used on a patient.

FIG. 3 depicts an example ambulatory medical device being used at another location on a patient.

FIG. 4 depicts an example process flow for determining an amount of thoracic fluid for a patient.

FIG. 5 depicts an example process flow for determining at least one weighting parameter.

FIG. 6 depicts an example adhesive patch.

FIG. 7 depicts an example monitoring unit.

FIG. 8 depicts an example of a monitoring unit being attached to an adhesive patch.

FIG. 9 depicts an example exploded view of a monitoring unit.

FIG. 10 depicts an example electronic architecture for a monitoring unit.

FIG. 11 depicts an example electronic architecture for RF functionality of a monitoring unit.

FIG. 12 depicts an example garment device.

FIG. 13 depicts an example electronic architecture for a medical device controller of a garment device.

### DETAILED DESCRIPTION

In some cases, a patient experiencing heart failure may simultaneously undergo a buildup of fluid in their lungs. The buildup occurs because the patient's heart cannot pump blood as well as it should, causing blood to back up into the blood vessels of the lungs. The backed-up blood may, in turn, leak into the interstitium and then the alveoli of the lungs. As such, in a cardiology practice, a caregiver may want to determine, measure, and/or monitor the amount of fluid in the patient's thorax, such as the percentage by volume of the patient's lungs that is filled with fluid. A patient may wear an ambulatory device capable of using RF waves to produce information relating to the amount of fluid in the patient's thorax. A processor may use the information from the RF waves to determine a relative amount of thoracic fluid, which can be tracked over time to determine whether the amount of fluid in the patient's thorax is increasing, decreasing, or staying the same. However, a caregiver may want to know the actual or absolute amount of thoracic fluid (e.g., as validated through a predetermined association with a predetermined “gold standard” metric), for example, as a percentage of the patient's thoracic fluid volume to the patient's lung volume. Knowing such actual or absolute amount of thoracic fluid in the patient's lungs may help the caregiver determine a treatment plan for the patient and/or evaluate the effectiveness of the patient's current treatment plan.

This disclosure relates to a patient monitoring system for improved thoracic fluid measurement in a patient. A patient is prescribed an ambulatory monitoring device configured to be worn continuously for an extended period of time. The monitoring device incorporates at least one RF antenna and associated RF circuitry configured to transmit RF waves towards a thoracic region of the patient. For example, the transmitted RF waves may be in a range of about 0.1 GHz to about 5 GHz. The at least one RF antenna and RF circuitry are further configured to receive RF waves reflected from the patient's thoracic region and/or RF waves transmitted through the patient's thoracic region, as well as generate at least one RF value from the received reflected and/or transmitted RF waves. As an example, RF circuitry may generate a matrix of values representing the amplitudes of received RF waves over time.

In implementations, at least one weighting parameter is formulated to output an amount of thoracic fluid in the patient wearing the ambulatory monitoring device from the RF values generated by an ambulatory monitoring device. Specifically, the at least one weighting parameter may be developed such that the output amount of thoracic fluid has a preconfigured association (e.g., correlation) with the patient's thoracic fluid as determined using a “gold standard” measurement technique, such as by computer tomography (CT) scans or by data from other clinical modalities for determining thoracic fluid, such as thermodilution, impedance, positron emission tomography (PET), magnetic resonance imaging (MRI), ultrasound, and/or the like. In this disclosure, systems, devices, and methods and/or computer-readable media are described to implement such data association analysis based on data assessed from clinical patient populations. For example, the at least one weighting parameter may be determined by a clinical modality process, such as a CT process that includes receiving reference CT scan-based fluid information for a patient population (e.g., normalized CT scan-based fluid information, such as normalized to account for differences in lung volume between patients of the patient population). As an illustration, a computing device, such as the remote server, a programmer device (e.g., implemented as a handheld device or a tablet carried by a medical care provider), or another computing device, may receive CT scans for a predetermined clinical patient population. The computing device operates in configurable electronic communication with a storage system such as a database, memory, or other non-transitory computer readable medium. In examples, in each CT scan, the areas of the patient's lungs that are filled with fluid are identified and compared to the total area of the patient's lungs. As such, the ratio of lung fluid to total lung area may be computed for each patient of the patient population. The CT process may also include producing RF-based thoracic fluid information for the patient population. As an illustration, the patients of the patient population may each wear an ambulatory monitoring device, and each ambulatory monitoring device may produce at least one RF value for the patient of the patient population that is wearing the ambulatory monitoring device. The computing device may then receive the RF values for the patient population. The RF-based thoracic fluid information may be the RF values for the patient population or may be the output of further processing applied to the RF values for the patient population.

The CT process may further include deriving the at least one weighting parameter using the RF-based thoracic fluid information and the reference CT scan-based thoracic fluid information. For example, in some implementations, the CT process may include optimizing an association, such as a degree of association, of the RF-based thoracic fluid information with the reference CT scan-based thoracic fluid information for the patient population. For example, such association or degree of association can include any relationship analysis between the RF-based thoracic fluid information and the reference CT scan-based thoracic fluid information. Measures of association can include correlation metrics or a combination of correlation metrics and difference metrics configured to depict a strength of the association between the RF-based thoracic fluid information and the reference CT scan-based thoracic fluid information. In this disclosure, while references are made to correlation analysis and/or processes, it is understood that other types of data association methods and/or statistical measures, e.g., Bland-Altman plots, can be used to measure a degree of association between the RF-based metrics and a chosen standard.

As an example, the computing device that has received the reference CT scan-based thoracic fluid information and the RF-based thoracic fluid information may chart a CT-scan based thoracic fluid metric for a patient (e.g., as determined using the reference CT scan-based thoracic fluid information), against the output of an RF-based thoracic fluid model that includes, as at least one input, an RF-based thoracic fluid metric (e.g., as determined using the RF-based thoracic fluid information) for the patient. In implementations, the RF-based thoracic fluid model may accept inputs in addition to the RF-based thoracic fluid metric. For example, such inputs may include patient health information for the patient of the patient population, such as demographic information (e.g., age, gender, height, etc. for the patient), biometric information (e.g., body mass index (BMI), heart rate, respiration rate, heart failure type, comorbidities, cardiac events, chest circumference, thoracic tissue thickness, etc. for the patient), and/or anthropomorphic information (e.g., height, weight, BMI, etc. for the patient). The computing device can chart the CT-scan based thoracic fluid metric against the output of the RF-based thoracic fluid model for each patient of the patient population and determine a correlation between the CT-scan based thoracic fluid metric and the output of the RF-based thoracic fluid model.

As illustrated in further detail below, during a tuning phase of operation, the computing device can modify or update the RF-based thoracic fluid model to optimize the correlation between the CT-scan based thoracic fluid metric and the output of the RF-based thoracic fluid model for the patient population. For example, the computing device can modify or update the inputs of the RF-based thoracic fluid model and/or one or more coefficients applied to the inputs of the RF-based thoracic fluid model. For example, the modification or update can occur iteratively. As an illustration, the modification or update can occur when new data is available or on a predetermined recurring basis, e.g., every second, every minute, every hour, or some other frequency set by a user-configurable parameter. Once the correlation is optimized in this manner during the tuning phase of operation, the computing device may set the one or more coefficients applied to the inputs of the RF-based thoracic fluid model as the at least one weighting parameter for use with a patient in the field.

The above process for producing the at least one weighting parameter is an example process. Other processes may be implemented without deviation from the spirit and scope of the disclosures. For example, in some implementations, the process for producing the at least one weighting parameter may correlate an RF-based thoracic fluid model with other sources of thoracic fluid information that can be used as a predetermined standard, such as thoracic fluid information determined from thermodilution, impedance, PET scans, MRI scans, ultrasounds, and/or the like.

Returning to the patient wearing the ambulatory medical device for an extended period of time, the patient monitoring system may further include a remote server in communication with the ambulatory medical device, where the remote server is configured to receive the at least one RF value from the ambulatory medical device. The remote server is further configured to retrieve the at least one weighting parameter from a memory of the remote server and apply the at least one weighting parameter to the at least one RF value to determine an amount of thoracic fluid in the patient and output the determined amount of thoracic fluid in the patient. In some implementations, the remote server is configured to apply the at least one weighting parameter directly to the at least one RF value (e.g., directly to the raw data received from the ambulatory medical device). In some implementations, the remote server is configured to process the at least one RF value and apply the at least one weighting parameter to the processed RF value data. As an illustration of the above, the remote server may retrieve the RF-based thoracic fluid model from the database of the remote server and apply the at least one weighting parameter of the RF-based thoracic fluid model to the at least one RF value (either directly or after processing by the remote server), along with any other necessary inputs for the RF-based thoracic fluid model. For example, the remote server may determine and/or receive (e.g., via user input) at least one patient health value for the patient, such as a demographic of the patient (e.g., age, gender, etc. for the patient), a biometric of the patient (e.g., BMI, heart rate, respiration rate, heart failure type, comorbidities, cardiac events, chest circumference, thoracic tissue thickness, etc. for the patient), and/or an anthropomorphic metric of the patient (e.g., height, weight, BMI, etc. for the patient). The remote server may then apply the at least one weighting value to the at least one RF value along with the at least one patient health value to determine the amount of thoracic fluid in the patient.

In one example use case, a caregiver may prescribe that a patient at risk of heart failure wear an ambulatory monitoring device for a certain amount of time (e.g., 15 days, 30 days, 60 days, 90 days). The ambulatory medical device is configured to be positioned over the patient's sternum such that the ambulatory medical device may transmit RF waves into the patient's thorax and receive RF waves reflected from the patient's thorax and/or transmitted through the patient's thorax, where the received RF waves contain information about a thoracic fluid level in the patient. The ambulatory medical device may thus take periodic RF measurements over the prescribed time of wear. For instance, the ambulatory medical device may take periodic RF measurements based on some or all of the following:
a predetermined schedule (e.g., at the same time every hour, at the same time every few hours, at the same time every day, a few determined times a week, and so on); this feature may be user-configurable;
when the patient is determined to be stationary (e.g., using data from an accelerometer in the ambulatory medical device) and/or when the patient is determined to be sleeping (e.g., using data from an accelerometer and/or time-of-day information);
the measurement falls within an expected range of values (e.g., if the values are outside the range, the device may discard or re-take the measurement);
when a predetermined triggering event occurs, such as a respiratory event (e.g., apnea, hypopnea, hyperpnea, and/or other breathing difficulties), a cardiac event based on ECG, and/or cardiovibrational data (e.g., atrial fibrillation, bradycardia, asystole, pause, murmurs, etc.); and/or
when a user trigger is received (e.g., patient reporting a symptom, such as shortness of breath, chest pain, heart flutter, etc.).

The ambulatory monitoring device may transmit the RF measurements (e.g., including at least one RF value) to a remote server, which determines one or more amounts of thoracic fluid in the patient using the at least one weighting value. The remote server may also prepare one or more reports for the caregiver and/or a technician to review. For instance, the remote server may prepare an end-of-use report for the caregiver that includes changes in the amount of thoracic fluid in the patient over the prescribed time of wear.

In another example use case, a caregiver may prescribe that a patient with cardiovascular issues wear an ambulatory monitoring device for a certain amount of time (e.g., 15 days, 30 days, 60 days, 90 days). The ambulatory medical device may include the at least one RF antenna and RF circuitry, as described above, along with additional sensors for monitoring the patient's cardiovascular health. For example, the ambulatory medical device may include one or more ECG electrodes configured to monitor ECG signals of the patient. As another example, the ambulatory medical device may include a motion sensor configured to monitor the patient's activity and/or position. The ambulatory medical device may take RF measurements, as well as additional measurements such as ECG measurements, motion measurements, position measurements, and so on, and transmit the measurements (e.g., including the at least one RF value) to a remote server. The remote server may determine one or more amounts of thoracic fluid in the patient over the prescribed wear period using the RF measurements and the at least one weighting value. In some implementations, the remote server may use the additional measurements to determine the patient's heart rate, respiration rate, posture, activity level, and so on over the prescribed wear period. In some implementations, the remote server may use the additional measurements to determine one or more arrhythmias that the patient experienced over the prescribed wear period. Additionally, in some implementations, the remote server may prepare one or more reports for the caregiver that summarize the one or more amounts of thoracic fluid determined by the remote server, along with any determined arrhythmias, heart rate information, respiration rate information, posture information, activity level information, and so on for the prescribed wear period.

In another example use case, a patient may be at high risk for a cardiac event (e.g., a life-threatening cardiac arrhythmia). As such, the patient's caregiver may prescribe that the patient continuously wear a garment-based ambulatory medical device until the patient is scheduled for a surgery to receive an implantable defibrillator. The patient may wear the garment-based ambulatory medical device (e.g., which may be shaped like a vest) while the medical device monitors the patient for a treatable arrhythmia via one or more sensing ECG electrodes. If a treatable arrhythmia is detected, the garment-based ambulatory medical device charges therapeutic electrodes that provide a shock to the patient. Additionally, the garment-based ambulatory medical device may include at least one RF antenna and RF circuitry, as described above, which produce at least one RF value for the patient that contains information about the patient's thoracic fluid level. The garment-based ambulatory medical device may transmit the at least one RF value to a remote server, which determines one or more amounts of thoracic fluid in the patient using the at least one RF value and at least one weighting parameter. The remote server may then alert the patient's caregiver if the remote server determines that the amount of thoracic fluid in the patient has reached a certain level, if the level of thoracic fluid in the patient has changed by a certain amount (e.g., increased by a predetermined percentage), and so on.

The patient monitoring system described herein may provide several advantages over prior art systems. In contrast to systems determining relative amounts of thoracic fluid metrics (e.g., to monitor for relative changes in the patient's thoracic fluid over time), patient monitoring systems described herein determine an amount of thoracic fluid that is highly correlated with a reference thoracic fluid measurement or information, such as normalized CT-scan thoracic fluid information for a patient population. For example, the high correlation may be set to be Pearson's coefficient, R, in a range between +0.7 to +1. In this regard, a user-configurable parameter may be used to adjust the correlation to be, e.g., within the range between +0.7 to +1 as described in further detail below. As such, the amount of thoracic fluid determined by the patient monitoring system is representative of an absolute or actual amount of thoracic fluid for the patient on the basis of such association measure. A caregiver for the patient may be able to use the amount or amounts of thoracic fluid output by the patient monitoring system to see if the patient has a low level of thoracic fluid, a normal level of thoracic fluid, or a high level of thoracic fluid. Such characterization is beneficial over systems that provide solely relative change information over time.

Additionally, in some implementations, the patient monitoring system described herein may provide the caregiver with changes to the amounts of the thoracic fluid over time. Such feature(s) provides information of not only whether the patient has low, normal, or high current levels of thoracic fluid but also, over time, changes to those levels of the thoracic fluid. In this regard, a caregiver may take different treatment actions when provided with changing amounts of thoracic fluid over time that are representative of absolute amounts of thoracic fluid in the patient on the basis of the correlation measure. As an illustration, a caregiver may be concerned by an overall increase in relative amounts of thoracic fluid over time, but the caregiver may not be concerned by an overall increase in a patient's thoracic fluid level if the patient is shown to have a low starting level of thoracic fluid by the amount of thoracic fluid output by the patient monitoring system. In this illustration, the caregiver may make no changes to the patient's treatment plan based on the amounts of thoracic fluid output by the patient monitoring system. As another illustration, a caregiver may not be concerned by a stable level of relative thoracic fluid, but the caregiver may be concerned if the patient's thoracic fluid level remains the same where the patient is shown to have a high starting level of thoracic fluid by the amount of thoracic fluid output by the patient monitoring system. In this illustration, the caregiver may determine that the patient's treatment plan is not resulting in necessary improvements to the patient's cardiovascular health and may make modifications to the patient's treatment plan.

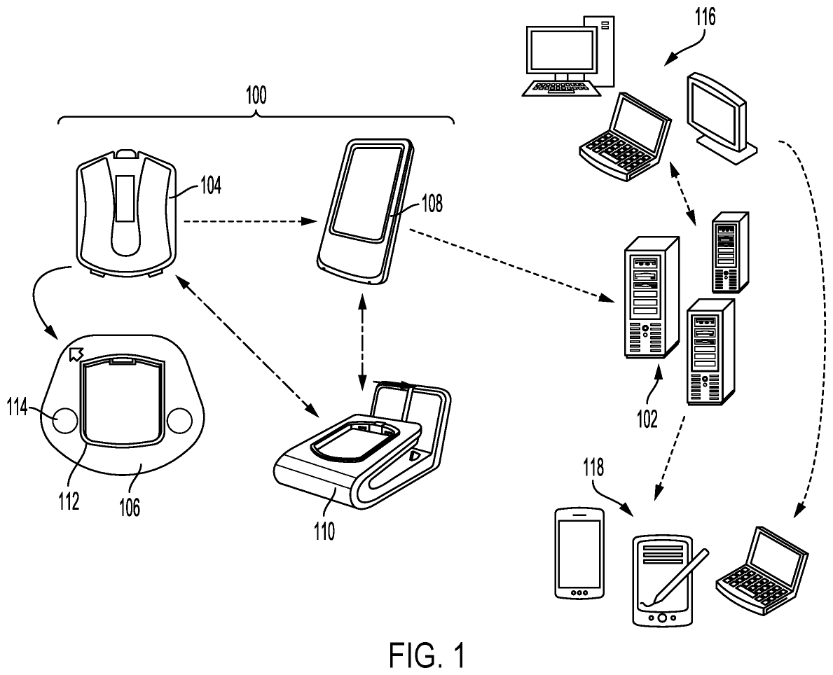

FIG. 1 shows a patient monitoring system that includes an ambulatory medical device 100 in communication with a remote server 102. The ambulatory medical device 100 is configured to provide at least one RF value that includes information about the patient's thoracic fluid level to the remote server 102. In some embodiments, the ambulatory medical device 100 may include and/or be at least partially implemented by the μCor™ Heart Failure Management System (HFMS) available from ZOLL® Medical Corporation of Chelmsford, MA. In some implementations, the ambulatory medical device 100 includes a monitoring unit 104, an adhesive patch 106, a portable gateway 108, and a charger 110. The monitoring unit 104 is configured to transmit RF waves into a thoracic region of a patient and receive RF waves reflected from the thoracic region and/or transmitted through the thoracic region. For example, the monitoring unit 104 may be a single unit configured to be positioned on the patient's thoracic region, with the monitoring unit 104 transmitting waves into the patient's thoracic region and receiving waves reflected back from the patient's thoracic region. As another example, the monitoring unit 104 may include a first portion configured to be positioned on one side of the thoracic region and a second portion configured to be positioned on the other side of the thoracic region. As such, the monitoring unit 104 may transmit RF waves into the thoracic region via the first portion and receive RF waves transmitted through the thoracic region via the second portion. In implementations, accordingly, the monitoring unit 104 includes at least one RF antenna and RF circuitry configured to transmit the RF waves into the patient's thoracic region and receive RF waves reflected from and/or transmitted through the patient's thoracic region. The monitoring unit 104 is further configured to use the received RF waves to generate at least one RF value therefrom, which the monitoring unit 104 transmits to the remote server 102. In some implementations, for example, the monitoring unit 104 may transmit the at least one RF value to the portable gateway 108, which transmits the at least one RF value to the remote server 102.

The adhesive patch 106 is configured to be adhesively coupled to the skin of the patient. The adhesive patch 106 is further configured such that the monitoring unit 104 may be removably attached to the adhesive patch 106. For example, the adhesive patch 106 may include a frame 112 in the same general shape of the monitoring unit 104, and the monitoring unit 104 may removably couple, connect, or snap into the frame 112. In some implementations, at least one RF antenna and RF circuitry for transmitting and receiving RF waves may be wholly or partially implemented in the adhesive patch, rather than wholly implemented in the monitoring unit 104. For example, the adhesive patch 106 may include the at least one RF antenna and RF circuitry, as well as electrical contacts to electrically connect the adhesive patch 106 to the monitoring unit 104. As such, the RF antenna and RF circuitry may receive RF waves from the thoracic region of the patient's body, which the monitoring unit 104 stores.

In some implementations, the monitoring unit 104 and/or adhesive patch 106 may include one or more additional sensors configured to sense other biometric signals of the patient. For instance, two or more ECG electrodes 114 may be embedded into the adhesive patch 106. In examples, the two or more ECG electrodes 114 may be instead partially or wholly disposed on the bottom surface of the monitoring unit 104. Regardless of the implementation of the ECG electrodes 114, the monitoring unit 104 may receive signals from the ECG electrodes 114 indicative of the ECG of the patient. As another illustration, the monitoring unit 104 may include a motion sensor configured to generate motion signals associated with the patient, where the motion signals include information about the activity and/or position (e.g., posture) of the patient. Examples of a motion sensor may include a 1-axis channel accelerometer, 2-axis channel accelerometer, 3-axis channel accelerometer, multi-axis channel accelerometer, gyroscope, magnetometer, ballistocardiograph, and the like. In some implementations, the portable gateway 108 may alternatively or additionally include one or more additional sensors configured to sense other biometric signals of the patient. For example, the portable gateway 108 may include a 3-axis accelerometer configured to generate motion signals, including activity and/or position information, associated with the patient.

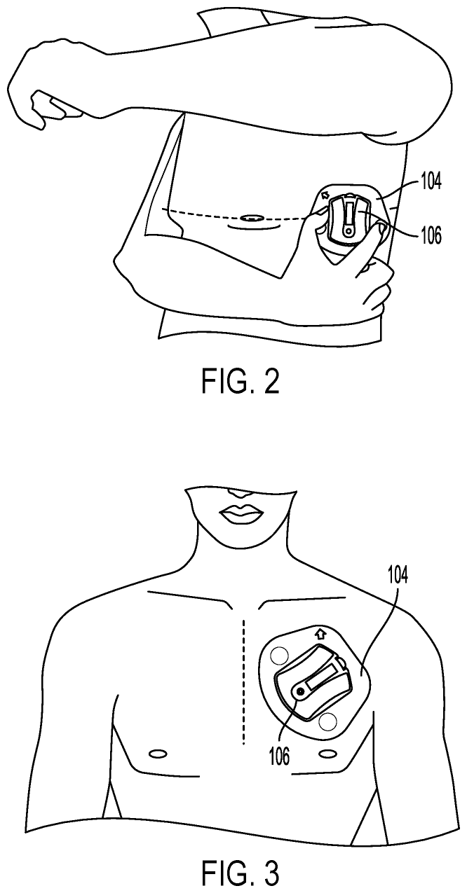

FIGS. 2 and 3 show examples of locations on the body of a patient where an adhesive patch 106 housing a monitoring unit 104 may be placed. As an example, the adhesive patch 106 may be placed on the side of the patient's thorax (e.g., under the patient's armpit, level with a circumferential line running across the patient's chest), as illustrated in FIG. 2. As another example, the adhesive patch 106 may be placed on the front of the patient's thorax (e.g., to the left of a medial line running between the patient's collarbone, for patients without dextrocardia), as illustrated in FIG. 3. However, the adhesive patch 106 may also be placed on any part of the patient's thorax that allows for efficient monitoring and recording of physiological data that includes RF waves received from the patient's thorax and containing information about the patient's thoracic fluid level.

The monitoring unit 104 and adhesive patch 106 are configured for long-term and/or extended use or wear by, or attachment or connection to, a patient. For example, devices as described herein are capable of being continuously used or continuously worn by, or attached or connected to, a patient without substantial interruption (e.g., for 24 hours, 2 days, 5 days, 7 days, 2 weeks, 30 days or 1 month, or beyond such as multiple months or even years). In some implementations, such devices may be removed for a period of time before use, wear, attachment, or connection to the patient is resumed. As an illustration, the monitoring unit 104 may be removed for charging, to carry out technical service, to update the device software or firmware, for the patient to take a shower, and/or for other reasons or activities without departing from the scope of the examples described herein. As another illustration, the patient may remove a used adhesive patch 106, as well as the monitoring unit 104, so that the patient may adhere a new adhesive patch 106 to their body and attach the monitoring unit 104 to the new adhesive patch 106. Such substantially or nearly continuous use, monitoring, or wear as described herein may nonetheless be considered continuous use, monitoring, or wear.

Returning to FIG. 1, as discussed above, the portable gateway 108 is configured to receive the signals provided by the monitoring unit (e.g., signals encoding the at least one RF value) and transmit the signals to the remote server 102. Accordingly, the portable gateway 108 may be in wired and/or wireless communication with the monitoring unit 104 and the remote server 102. As an illustration, the portable gateway 108 may communicate with the monitoring unit 104 via Ethernet, via Wi-Fi, via near-field communication (NFC), via radiofrequency, and the like. The portable gateway 108 may further communicate with the remote server 102 via cellular networks, via Bluetooth®-to-TCP/IP access point communication, via Ethernet, via Wi-Fi, and the like. As such, the portable gateway 108 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the monitoring unit 104 and/or the portable gateway 108 may be part of an Internet of Things (IOT) and communicate with each other and/or the remote server 102 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

Alternatively, in some implementations, the monitoring unit 104 may be configured to transmit the sensed or acquired signals (e.g., the at least one RF value) directly to the remote server 102 instead of, or in addition to, transmitting the signals to the portable gateway 108. Accordingly, the monitoring unit 104 may be in wired or wireless communication with the remote server 102. As an illustration, the monitoring unit 104 may communicate with the remote server 102 via cellular networks, via Bluetooth®-to-TCP/IP access point communication, via Ethernet, via Wi-Fi, and the like. Further, in some implementations, the ambulatory medical device 100 may not include the portable gateway 108. In such implementations, the monitoring unit 104 may perform the functions of the portable gateway 108 described above. Additionally, in such implementations, the monitoring unit 104 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, as indicated above, the communications circuitry in the monitoring unit 104 may be part of an IoT and communicate with the remote server 102 via IoT protocols for handling secure (e.g., encrypted) messaging and routing.

The charger 110 includes charging cradles configured to hold and recharge the monitoring unit 104 and the portable gateway 108. Alternatively, in some implementations, the ambulatory medical device 100 may not include the portable gateway 108, and accordingly, the charger 110 may be configured to hold the monitoring unit 104 alone. In some implementations, the monitoring unit 104 and the portable gateway 108 may have separate chargers, or one or both of the monitoring unit 104 and the portable gateway 108 may have removable batteries that may be replaced and/or recharged.

The remote server 102 is configured to receive and process the signals transmitted by the ambulatory medical device 100. Accordingly, the remote server 102 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the signals transmitted by the ambulatory medical device 100. For example, the at least one processor of the remote server 102 may be configured similarly to the processor 1318 of the garment device 1200 discussed in further detail below. For instance, the at least one processor of the remote server 102 may be implemented as a digital signal processor (DSP), such as a 24-bit DSP processor; as a multicore-processor (e.g., having two or more processing cores); as an Advanced RISC Machine (ARM) processor, such as a 32-bit ARM processor; and/or the like. The at least one processor of the remote server 102 can execute an embedded operating system and further execute services provided by the operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others. In various implementations, the remote server 102 may use the data transmitted by the ambulatory medical device 100 (e.g., including at least one RF value for the patient wearing the ambulatory medical device 100) to determine one or more amounts of thoracic fluid for the patient, as described in further detail below. Alternatively, in some implementations, the ambulatory medical device 100 may determine the one or more amounts of thoracic fluid (e.g., at the monitoring unit 104 and/or the portable gateway 108) and transmit the one or more amounts of thoracic fluid to the remote server 102 (e.g., via the portable gateway 108).

As shown in FIG. 1, in some implementations, the patient monitoring system further includes one or more user interfaces, such as technician interfaces 116 and caregiver interfaces 118. The technician interfaces 116 and caregiver interfaces 118 are in electronic communication with the remote server 102 through a wired or wireless connection. For instance, the technician interfaces 116 and caregiver interfaces 118 may communicate with the remote server 102 via Wi-Fi, via Ethernet, via cellular networks, and/or the like. Additionally, as shown, at least some of the technician interfaces 116 may be in electronic communication with at least some of the caregiver interfaces 118 through a wired or wireless connection, such as via Wi-Fi, via Ethernet, via cellular networks, and/or the like. The technician interfaces 116 and the caregiver interfaces 118 may include, for example, desktop computers, laptop computers, and/or portable personal digital assistants (e.g., smartphones, tablet computers, etc.).

In some implementations, the technician interfaces 116 are configured to electronically communicate with the remote server 102 for the purpose of viewing and analyzing information gathered from one or more ambulatory medical devices 100 (e.g., at least one RF value for a patient wearing an ambulatory medical device 100, ECG data for a patient wearing an ambulatory medical device 100, and so on). For example, a technician interface 116 may provide one or more instructions to the remote server 102 to prepare a summary report of the data for a patient (e.g., including one or more amounts of thoracic fluid determined for the patient) for a certain time period. Accordingly, a technician interface 116 may include a computing device having a processor communicably connected to a memory and a visual display. The technician interface 116 may display to a user of the technician interface 116 (e.g., a technician) data gathered from one or more ambulatory medical devices 100 and/or information computed from the data gathered from the one or more ambulatory medical devices 100 (e.g., an amount of thoracic fluid for a patient determined from at least one RF value received for the patient). The user of the technician interface 116 may then provide one or more inputs to the remote server 102 to guide the remote server 102 in preparing a report for a patient.

As an example, a user of a technician interface 116 may select a time period to use for a report, and the remote server 102 may prepare a report corresponding to the selected time period. As another example, a user of a technician interface 116 may select types of data to be included in a report, such as an initial amount of lung fluid, lung fluid amounts over time, arrhythmia information, heart rate information, respiratory rate information, sleep information, and so on for a patient. The remote server 102 may thus prepare a report according to the types of data selected by the user. As another example, a user of a technician interface 116 may view a report prepared by the remote server 102 and draft a summary of the report to be included in a summary section for the report. Alternatively, in some implementations, the remote server 102 may analyze and/or summarize the data gathered from the one or more ambulatory medical devices 100 with minimal or no input or interaction with a technician interface 116. In this way, the remote server 102 may analyze and/or summarize the information gathered from the one or more ambulatory medical devices 100, and prepare a report on this information, through a completely or mostly automated process.

The caregiver interfaces 118 are configured to electronically communicate with the remote server 102 for the purpose of viewing information on patients wearing ambulatory medical devices 100. As such, a caregiver interface 118 may include a computing device having a processor communicably connected to a memory and a visual display. The caregiver interface 118 may display to a user of the caregiver interface 118 (e.g., a physician, a nurse, or other caregiver) one or more amounts of thoracic fluid determined for a patient, ECG waveforms for a patient, arrhythmia information for a patient, health and biometric information for a patient, and/or the like. In some implementations, the caregiver interface 118 may display to a user one or more reports summarizing thoracic fluid information, ECG information, arrhythmia information, health and biometric information, and/or the like for a patient, such as reports prepared by the remote server 102 (e.g., based on inputs from the one or more technician interfaces 116). In some implementations, the user of a caregiver interface 118 may be able to interact with the information displayed on the caregiver interface 118. For example, the user of a caregiver interface 118 may be able to select a portion of a patient report and, in response, be able to view additional information relating to the selected portion of the report. Additional information may include, for instance, ECG waveforms used to identify arrhythmias, amounts of thoracic fluid for the patient over time, an initial amount of thoracic fluid for the patient, a health and biometric context for the patient (e.g., whether the patient was sleeping, active, had normal respiration, and so on for a selected time in the report), and/or the like. In some implementations, the user of the caregiver interface 118 may instead view a static patient report that does not have interactive features.

In some implementations, a technician interface 116 and/or a caregiver interface 118 may be a specialized user interface configured to communicate with the remote server 102. As an example, the technician interface 116 may be a specialized computing device configured to receive preliminary patient reports from the remote server 102, receive inputs from a user to adjust the preliminary report, and transmit the inputs back to the remote server 102. The remote server 102 then uses the inputs from the technician interface 116 to prepare a finalized patient report, which the remote server 102 also transmits to the technician interface 116 for review by the user. As another example, the caregiver interface 118 may be a specialized computing device configured to communicate with the remote server 102 to receive and display patient reports, as well as other information for patients wearing an ambulatory medical device 100.

In some implementations, a technician interface 116 and/or a caregiver interface 118 may be a generalized user interface that has been adapted to communicate with the remote server 102. To illustrate, the technician interface 116 may be a computing device (e.g., a laptop, a portable personal digital assistant such as a smartphone or tablet, etc.) executing a technician application that configures the computing device to communicate with the remote server 102. For example, the technician application may be downloaded from an application store or otherwise installed on the computing device. Accordingly, when the computing device executes the technician application, the computing device is configured to establish an electronic communication link with the remote server 102 to receive and transmit information regarding patients wearing ambulatory medical devices 100. Similarly, the caregiver interface 118 may be a computing device (e.g., a laptop, a portable personal digital assistant such as a smartphone or tablet, etc.) executing a caregiver application that configures the computing device to communicate with the remote server 102. The caregiver application may be similarly downloaded from an application store or otherwise installed on the computing device and, when executed, may configure the computing device to establish a communication link with the remote server 102 to receive and display information on patients wearing ambulatory medical devices 100.

The application store is typically included within an operating system of a computing device implementing a user interface. For example, in a device implementing an operating system provided by Apple Inc. (Cupertino, California), the application store can be the App Store, a digital distribution platform, developed and maintained by Apple Inc., for mobile apps on its iOS and iPadOS® operating systems. The application store allows a user to browse and download an application, such as the technician or caregiver application, developed in accordance with the Apple® iOS Software Development Kit. For instance, such technician or caregiver application may be downloaded on an iPhone® smartphone, an iPod Touch® handheld computer, or an iPad® tablet computer, or transferred to an Apple Watch® smartwatch. Other application stores may alternatively be used for other types of computing devices, such as computing devices operating on the Android® operating system.

In some implementations, the technician application and the caregiver application may be the same application, and the application may provide different functionalities to the computing device executing the application based on, for example, credentials provided by the user. For instance, the application may provide technician functionalities to a first computing device in response to authenticating technician credentials entered on the first computing device, and may provide caregiver functionalities to a second computing device in response to authenticating caregiver credentials entered on the second computing device. In other cases, the technician application and the caregiver application may be separate applications, each providing separate functionalities to a computing device executing them.

In some implementations, the patient monitoring system shown in FIG. 1 may include other types of interfaces. To illustrate, in some examples, the patient monitoring system may include patient interfaces. Thus, the remote server 102 and/or a technician interface 116 may provide a report on a patient wearing an ambulatory medical device 100 to this patient via a patient interface. This patient report may be the same as a report provided to a caregiver via a caregiver interface 118, or this patient report may be different from the report provided to the caregiver via the caregiver interface 118. For instance, the report provided to a patient may be an abridged version of a report prepared for the patient's caregiver, such as a report that summarizes the key information from the report prepared for the patient's caregiver. In various implementations, the patient interface may be configured similarly to and function similarly to the caregiver interface 118 discussed above (e.g., with some additional restrictions on what is included in a patient report compared to a report prepared for the patient's caregiver and/or on the functionalities of, for instance, an application executed by the patient interface that the patient can use to access and view a patient report or other information on the patient).

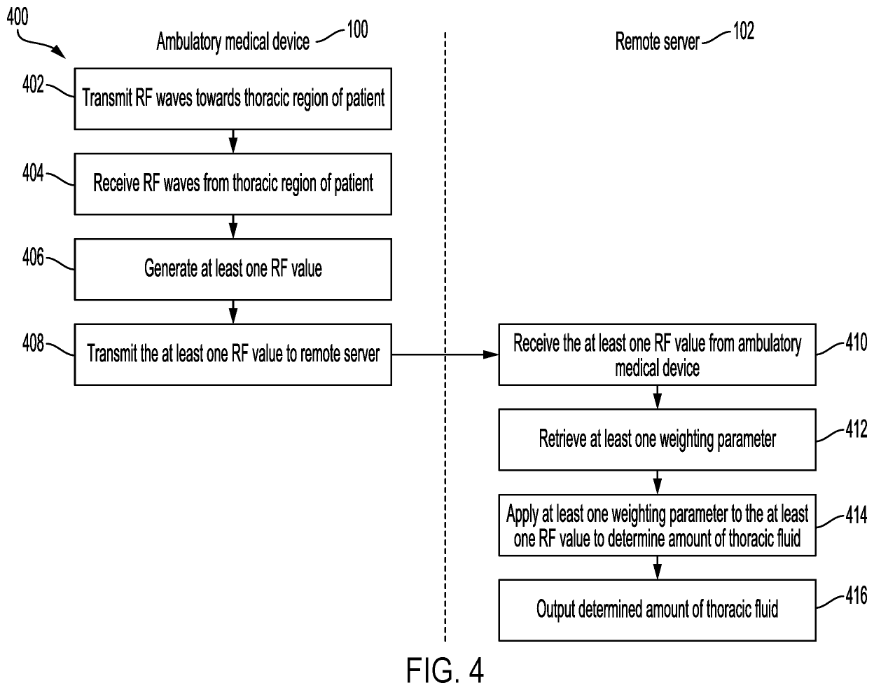

FIG. 4 illustrates a sample process flow for determining an amount of thoracic fluid for a patient. The sample process 400 shown in FIG. 4 can be implemented by an ambulatory medical device 100 (e.g., by the monitoring unit 104 independently or in concert with the portable gateway 108) and a remote server 102. As shown in FIG. 4, an ambulatory medical device 100 mounted on a thoracic region of the patient (e.g., the adhesive patch 106 of the ambulatory medical device 100 affixed to the thoracic region and the monitoring unit 104 attached to the adhesive patch 106) transmits RF waves toward the thoracic region at step 402. To illustrate, the monitoring unit 104 may generate and transmit RF waves towards the thoracic region of the patient via at least one RF antenna and associated RF circuitry contained in the monitoring unit 104.

The ambulatory medical device 100 then receives RF waves received from the thoracic region of the patient at step 404. In some implementations, the monitoring unit 104 of the ambulatory medical device 100 transmits the RF waves into the thoracic region of the patient and receives reflected RF waves transmitted through the thoracic region and/or RF waves back from the thoracic region. The transmitted RF waves may be, for example, in a range of 0.1 GHz to 5.0 GHz or a narrower range, such as 0.5 GHz to 2.1 GHZ. The RF measurements may be taken in RF measurement sessions lasting about 30 seconds, about 1 minute, about 2 minutes, about 3 minutes, about 4 minutes, about 5 minutes, about 10 minutes, and so on (e.g., plus or minus a predetermined amount, such as 5-15% or 10-30 seconds). In examples, there may be a preconfigured number of RF measurement sessions in a preconfigured duration of time. For example, there may be a preconfigured number of RF measurement sessions in an hour, two hours, one day, one week, or the like. For example, there may be 20 RF measurement sessions in the predetermined duration of time, 50 RF measurement sessions, 100 RF measurement sessions, 200 RF measurement sessions, or more. The number of RF measurement sessions and period may each be adjusted via a user-configurable parameter.

In some implementations, RF measurements may be discarded if the patient shows undesirable positioning and/or motion during the sampling period. For example, the ambulatory medical device 100 and/or remote server 102 may identify whether the patient showed movement above a predetermined threshold while the RF sampling was taking place (e.g., based on counts by an accelerometer in the ambulatory medical device 100, such as the accelerometer 1022 discussed below). If the patient showed movement transgressing a predetermined threshold, the ambulatory medical device 100 and/or remote server 102 may discard the RF measurements. As another example, the ambulatory medical device 100 and/or remote server 102 may identify the patient's body posture (e.g., degree of orientation) while the RF sampling was taking place (e.g., as measured by an accelerometer in the ambulatory medical device 100, such as the accelerometer 1022 discussed below). If the patient's body posture is close or substantially close to a target posture (e.g., within 5% of the target posture, within 10% of the target posture, within 15% of the target posture, etc.), the ambulatory medical device 100 and/or remote server 102 may keep the measurement. Otherwise, the ambulatory medical device 100 and/or remote server 102 may discard the measurement. In some examples, if a measurement is discarded, the ambulatory medical device 100 and/or remote server may reschedule the measurement, for a user-configurable duration, for example, between 1 to 10 minutes, 1 minute to 1 hour, or 1 hour to 1 day in the future. In examples, the timing for rescheduling the measurement may be a configurable parameter having a default, for example, of 3 minutes.

In some implementations, as indicated above, the monitoring unit 104 of the ambulatory medical device 100 transmits the RF waves into the thoracic region of the patient and receives RF waves transmitted through the thoracic region. In such implementations, the ambulatory medical device 100 may include a secondary adhesive patch configured to be adhered on the opposite side of the patient's thoracic region from the adhesive patch 106. The monitoring unit 104 may be further split into a primary unit section, including a first at least one RF antenna and first RF circuitry, configured to attach to the adhesive patch 106, and a secondary unit section, including a second at least one RF antenna and second RF circuitry, configured to attach to the secondary adhesive patch. Alternatively, the ambulatory medical device 100 may not include a secondary adhesive patch, and the secondary unit section may be configured to attach directly to the patient's thoracic region (e.g., via a permanent or replaceable adhesive section on the secondary unit section). The primary unit section may transmit the RF waves towards the thoracic region of the patient, and the secondary unit section may receive the RF waves transmitted through the patient's thoracic region. The secondary unit section may be further configured to communicate (e.g., wirelessly or via a wired connection) with the primary unit section and/or the portable gateway 108 to transmit signals that include information about the received RF waves.

In some implementations, the ambulatory medical device 100 may be configured to include a single monitoring unit 104 as shown in FIG. 1. The monitoring unit 104 may be attached to the adhesive patch 106 (e.g., as shown in FIGS. 2 and 3), and may further transmit RF waves towards the thoracic region of the patient and receive RF waves reflected from the thoracic region. In some implementations, the ambulatory medical device 100 may both receive reflected RF waves and RF waves transmitted through the patient's thoracic region. As such, the ambulatory medical device 100 may be configured as a hybrid of the examples provided above.

The ambulatory medical device 100 generates at least one RF value at step 406. For example, the ambulatory medical device 100 may record amplitudes of received RF waves in a matrix (e.g., at the monitoring unit 104 and/or the portable gateway 108), where the at least one RF value is the matrix of received RF wave amplitudes. To illustrate, when taking an RF measurement, the ambulatory medical device may transmit bursts of RF waves in a span of frequencies (e.g., a burst of waves in the 0.1 to 5.0 GHz range in at least 50 frequency steps). Thus, the RF measurement matrix may include rows, where each row represents a single RF stepped frequency measurement, and columns, where each column represents a single frequency step. For example, an RF measurement matrix may include 1000-5000 rows (e.g., 50 per second), with each row representing the burst number, and 50-100 columns, with each column representing the frequency number (e.g., from 1 to 50-100). In some implementations, the ambulatory medical device 100 may also generate a calibration vector. For instance, the ambulatory medical device 100 may take a calibration measurement prior to each RF measurement such that each element in the calibration vector refers to one of the transmitted RF frequencies. The calibration vector may represent the internal signal path and may be used to compensate for an internal sensor frequency response.

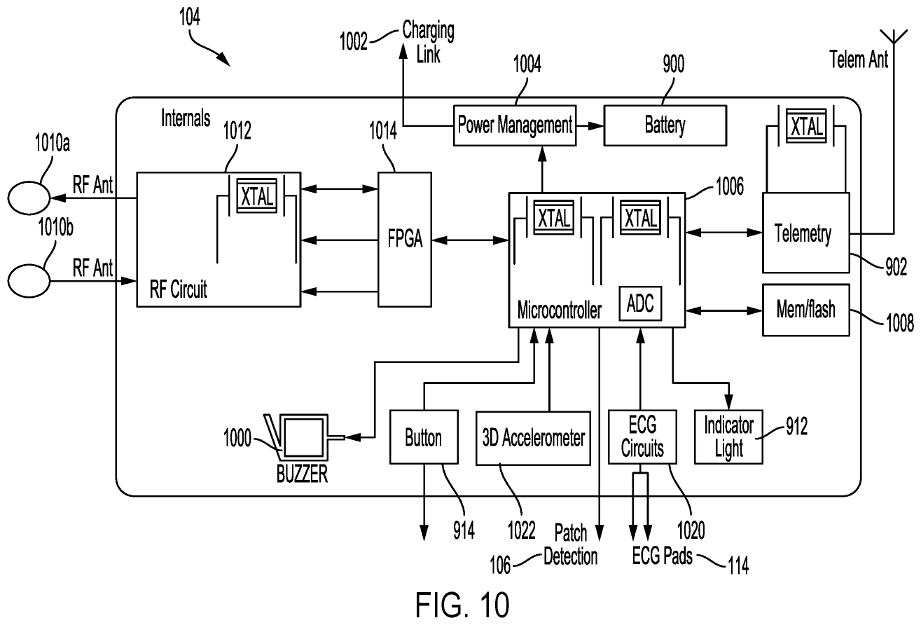

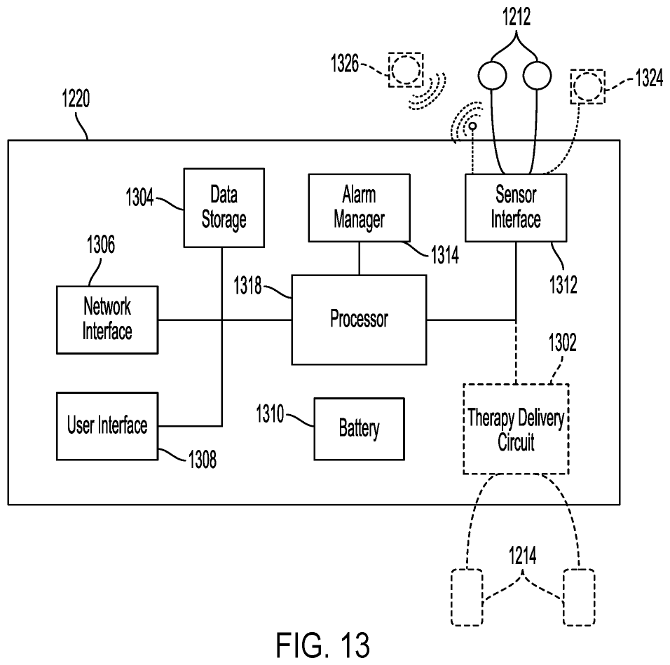

The ambulatory medical device 100 then transmits the at least one RF value (e.g., the RF measurement matrix) to the remote server 102 at step 408. As shown in FIG. 4, the remote server 102 receives the at least one RF value from the ambulatory medical device 100 at step 410. The associated hardware, devices, and systems configured to provide for the transmission of the at least one RF value to the remote server are described above in connection with FIG. 1 and further with reference to FIGS. 10 and 13 below.

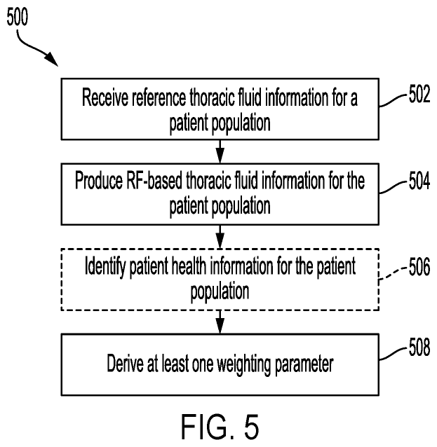

The remote server 102 retrieves at least one weighting parameter from a memory at step 412. A sample clinical modality process flow for determining the at least one weighting parameter (e.g., a CT process) is illustrated in FIG. 5 and will be described now before returning to the rest of the process of FIG. 4. While FIG. 5 depicts a CT scan-based process, it should be noted that the process of FIG. 5 can be modified to apply to thermodilution process-based data, impedance process-based data, PET process-based data, MRI process-based data, ultrasound process-based data, and/or the like. The sample process 500 shown in FIG. 5 can be implemented by a computing device or networked computing devices, such as the remote server 102 or another computing device/networked computing device including at least one processor in communication with a memory (e.g., with the memory storing sequences of instructions that can be executed by the processor to perform functions).

The computing device receives reference thoracic fluid information for a patient population at step 502. The reference thoracic fluid information for the patient population may be based on information from a clinically-used modality for measuring thoracic fluid. As an example, in some implementations, the computing device may receive CT scans of the thoracic region of each individual within the patient population. The computing device may then mark sections of the CT scans that represent thoracic fluid in a process that is normalized across all of the individuals of the patient population. The computing device may further determine a thoracic fluid volume or ratio (e.g., ratio of thoracic fluid volume to total thoracic volume) using the marked sections of CT scans. An example CT scan of a patient with thoracic fluid is shown in FIG. 2 of Gluecker et al., “Clinical and Radiologic Features of Pulmonary Edema,” 19 RadioGraphics 1507(1999), which is hereby incorporated by reference. The arrow in FIG. 2 points to “inter-and intralobar septal lines predominating in the anterior portion of the left lung field with some peribronchial chuffing” as a result of thoracic fluid.

As an illustration, the computing device may determine reference (e.g., normalized) extravascular thoracic fluid from the CT scans using the segmentation processes described in Fermoyle et al., “Assessment of Thoracic Blood Volume by Computed Tomography in Patients with Heart Failure and Periodic Breathing,” 24 J. Card. Fail. 479 (2018) and Chase et al., “Influence of Thoracic Fluid Compartments on Pulmonary Congestion in Chronic Heart Failure,” 23 J. Card. Fail. 690 (2017), which are hereby incorporated by reference. In this regard, with respect to determining extravascular lung water (“EVLW”) Fermoyle et al, states that “[t] he presence of EVLW was determined from thoracic CT scans. Lung tissue was segmented from adjacent tissues and large blood vessels using MATLAB built-in active contour algorithms (Mathworks, Inc, Natick, MA). Only pixels within the attenuation range of air (−1000 HU) and water (0 HU) were included in the analysis. The mean lung density was calculated from the attenuation distribution within the segmented areas. EVLW was calculated as fluid content (FD) from the mean lung density (MLD) as FC= (MLD+1000)/10.”

In some implementations, the reference thoracic fluid information for the patient population may exclude intravascular thoracic fluid information. In such implementations, the reference thoracic fluid information for the patient population may not include fluid that is in the patient's vasculature, such as fluid in the patient's pulmonary arteries. As such, the reference thoracic fluid information for the patient population may include, for example, information on the extravascular lung fluid for each individual of the patient population. Excluding the intravascular lung fluid information may be done so as to isolate the lung fluid due to the patient's potential heart failure status, as intravascular lung fluid will be present regardless of the heart failure status of the patient. Alternatively, in some implementations, the reference thoracic fluid information for the patient population may include both intravascular and extravascular lung fluid information. As such, if the thoracic area of interest is the patient's lungs, for instance, the reference thoracic fluid information may include reference extravascular and intravascular lung fluid information for the individuals of the patient population. In some implementations, the reference thoracic fluid information for the patient population may exclude thoracic fluid in the patient's pleural space (e.g., the space of the pleural cavity of the patient's lungs). Thus, the reference thoracic fluid information for the patient population may include non-pleural space thoracic fluid. In some implementations, the reference thoracic fluid information for the patient population may include thoracic fluid in the patient's pleural space.

Although step 502 is described above with respect to reference CT scan-based thoracic fluid information, in some implementations, the reference thoracic fluid information may instead be derived from a difference source. For example, in some implementations, the reference thoracic fluid information may be reference thermodilution-based thoracic fluid information. A caregiver may inject an indicator into the circulation of patients of a patient population and then measure the downstream concentrations of the indicator. Thermodilution may be performed as a double indicator method where, as an illustration, the patient may be given an intravenous (IV) infusion of cold saline and indocyanine green (ICG). The cold saline diffuses throughout the thoracic fluid space, while the ICG remains intravascular. By determining the volume of distribution of the cold saline and the ICG, the patient's extravascular thoracic fluid level can be estimated. Thermodilution may alternatively be performed as a single indicator method. As an illustration, a patient may be injected with a certain amount (e.g., 10-20 mL) of cold saline, and the patient's extravascular thoracic fluid level can be estimated by determining the volume of distribution of the cold saline, as well as using some assumptions about the volume of blood in the lung vessels.

As another example, in some implementations, the reference thoracic fluid information may be reference impedance-based thoracic fluid information. Impedance measurements of a patient's thoracic region may be taken using an implanted device or an external device. Using an implanted device is more invasive, but measurements taken with an external device may be affected by skin-to-electrode impedance. Impedance measurements of the patient's thoracic region may then be used to determine the total amount of thoracic fluid (e.g., total pulmonary fluid) in the patient's thoracic region.

As another example, in some implementations, the reference thoracic fluid information may be reference PET-based thoracic fluid information. A patient is administered tracers that are labelled with a positron-emitting isotope, and the emissions from these tracers are detected, such as by a gamma camera. The emissions can then be reconstructed to form a PET image. For the purpose of detecting thoracic fluid, a patient may be administered one or two tracers, depending on whether the total thoracic fluid or extravascular thoracic fluid is desired. As an illustration, a patient may be injected intravenously with H₂¹⁵O, and the total thoracic fluid volume can be calculated from the tracer activity within the thoracic region, the tracer activity in the blood in the heart, and the free water content in the blood. As another illustration, the patient may also inhale C¹⁵O, which binds to hemoglobin. Thus, the intravascular thoracic fluid volume can be calculated from the C¹⁵O tracer activity, and the extravascular thoracic fluid volume can be determined by subtracting the intravascular thoracic fluid volume from the total thoracic fluid volume.

As another example, in some implementations, the reference thoracic fluid information may be reference magnetic resonance imaging (MRI)-based thoracic fluid information. To illustrate, multi-echo sequences may be used to plot a signal decay curve, which can then be used to estimate water content by extrapolating the curve to zero echo time and comparing to a substance with a known water density. Further details on using thermodilution, impedance, PET, and MRI to determine thoracic fluid information are described in Gupta et al., “Methods of Measuring Lung Water,” 13 J. Intensive Care Soc. 209 (2012), which is hereby incorporated by reference.

As another example, in some implementations, the reference thoracic fluid information may be reference ultrasound-based thoracic fluid information. Reverberation artifacts called B-lines may be present in an ultrasound if the patient has interstitial thoracic fluid. As such, the number of B-lines may be indicative of the degree of thoracic fluid. Further details on using ultrasound to determine thoracic fluid information are described in Boorsma et al., “Congestion in Heart Failure: A Contemporary Look at Physiology, Diagnosis and Treatment,” 17 Nature Reviews 641 (2020) and Platz et al., “Expert Consensus Document: Reporting Checklist for Quantification of Pulmonary Congestion by Lung Ultrasound in Heart Failure,” 21 Eur. J. Heart Fail. 844 (2019), which are hereby incorporated by reference.

The computing device may further produce RF-based thoracic fluid information for the patient population at step 504. In some implementations, the individuals of the patient population may all be provided with an ambulatory medical device 100, and the computing device may receive RF values from the ambulatory medical devices 100 worn by the individuals of the patient population. For example, the ambulatory medical devices 100 may each send a matrix of RF values representing the amplitude of received RF waves for the respective individuals of the patient population, similar to the process described above with respect to step 404 of FIG. 4. The computing device may thus use the RF values from the ambulatory medical devices 100 to produce RF-based thoracic fluid information for the patient population. In some examples, the computing device may use the raw RF values from the ambulatory medical devices 100 as the RF-based thoracic fluid information. In some examples, the computing device may process the raw RF values from the ambulatory medical devices 100 to produce the RF-based thoracic fluid information, as discussed in further detail with respect to step 508 below.

In some implementations, the computing device may also identify patient health information for the patient population at step 506, though in some implementations of sample process 500, step 506 may be omitted, which is why step 506 is marked in dotted lines. In implementations of the sample process 500 including step 506, patient health information may include demographic information for the patient population (e.g., information describing physical attributes of the individuals of the patient population, such as age and height), biometric information for the patient population (e.g., information about the unique physiological and/or behavioral characteristics of the patient population, such as BMI, heart rate, and respiration rate), anthropomorphic information for the patient population (e.g., information about the physiological characteristics of the patient population, such as height, weight, and BMI), and/or the like. The metric classified as demographic information, biometric information, and anthropomorphic information may overlap, in some cases. In some implementations, the patient health information for the patient population includes at least one of gender information, age information, height information, weight information, BMI information, heart rate information, respiration rate information, heart failure type information, comorbidity information, cardiac events information (e.g., information about the types of arrhythmias experienced by the patient population), chest circumference information, thoracic thickness information, activity rate information, posture information, and/or the like for the patient population.

Identifying the patient health information for the patient population may include, in some implementations, receiving the patient health information as a user input (e.g., from one or more caregivers of the patient population) and/or determining the patient health information from signals recorded from the patient population that include patient health information. For example, the computing device may receive ECG signals for the patient population (e.g., recorded by the ambulatory medical devices 100 worn by the patient population) and identify heart rate and cardiac events information from the ECG signals for the patient population. As another example, the computing device may receive motion signals for the population (e.g., recorded by the ambulatory medical devices 100 worn by the patient population) and identify respiration rate, posture, and activity rate information for the patient population from the motion signals. In some implementations, the patient health information may be a statistical measure (e.g., average, median, maximum, minimum, mode, etc.) of a series of patient health information, such as the average or median heart rate or respiration rate for each individual of the patient population over a predetermined period of time.

The computing device is configured to use at least the reference thoracic fluid information for the patient population and the RF-based thoracic fluid information for the patient population to derive the at least one weighting parameter at step 508. In some implementations, deriving the at least one weighting parameter includes optimizing an association (e.g., a correlation) of the RF-based thoracic fluid information with the reference thoracic fluid information for the patient population. For example, for each individual of the patient population, the computing device may determine the patient's percent thoracic fluid level from the reference thoracic fluid information for the patient. Separately, the computing device may set an RF-based thoracic fluid model where at least one input of the RF-based thoracic fluid model is the RF-based thoracic fluid information for the patient or derived from the RF-based thoracic fluid information for the patient. Each input of the model may be adjusted by one or more weighting parameters (e.g., where the weighting parameter is a coefficient of the input, an exponent for the input, a derivative for the input, etc.) and/or the function as a whole may be adjusted by one or more weighting parameters (e.g., where the weighting parameter is an amount added to the adjusted inputs). The computing device may plot each patient's percent thoracic fluid level against the output of the RF-based thoracic fluid model. The computing device may then adjust the at least one weighting parameter of the RF-based thoracic fluid model to optimize the correlation between the percent thoracic fluid values and the outputs of the RF-based thoracic fluid model. For instance, the correlation may be considered optimized once an R value for the plot reaches a predetermined level (e.g., +0.7 or above, +0.8 or above, +0.9 or above) or once the R value for the plot reaches a maximum value, for example, as determined by the computing device using machine learning. The at least one weighting parameter retrieved at step 412 of FIG. 4 may thus be the at least one weighting parameter from the optimized RF-based thoracic fluid model.

Examples of this thoracic fluid function defining weighting parameters are provided below, where the X values represent inputs to the thoracic fluid function and the A, B, C, etc. values represent weighting parameters:

$$\begin{matrix}Thoracic fluid = A * X + B \\ Thoracic fluid = {A}_{1} * {X}_{1} + {A}_{2} * {X}_{2} + B \\ Thoracic fluid = {A}_{1} * {X}_{1} + {A}_{2} * {X}_{2} + … + {A}_{n} * {X}_{n} + B \\ Thoracic fluid = {A}_{1} * {X}_{1} + {A}_{2} * {X}_{2} - B * {X}_{3} + C \\ Thoracic fluid = A * {X}^{B} + C \\ Thoracic fluid = {A}_{1} * {X}_{1}^{{B}_{1}} + {A}_{2} * {X}_{2}^{{B}_{2}} + C \\ Thoracic fluid = {A}_{1} * {X}_{1}^{{B}_{1}} + {A}_{2} * {X}_{2}^{{B}_{2}} + … + {A}_{n} * {X}_{n}^{{B}_{n}} + C \\ Thoracic fluid = A * log ( X ) + B \\ Thoracic fluid = {A}_{1} * log ( {X}_{1} ) + {A}_{2} * log ( {X}_{2} ) + … + {A}_{n} * log ( {X}_{n} ) + B \\ Thoracic fluid = A * {e}^{X} + C \\ Thoracic fluid = A * {e}^{B * X} + C \\ Thoracic fluid = A * {e}^{{B}_{1} * {X}_{1} + {B}_{2} * {X}_{2} + … + {B}_{n} * {X}_{n}} + C\end{matrix}$$

Other examples of the thoracic fluid function defining weighting parameters may include nth-order polynomial functions, logarithmic functions, exponential functions, power functions, and/or the like, as well as combinations of these functions.

In some implementations, as discussed above, the RF-based thoracic fluid information used in the RF-based thoracic fluid model may not be the raw RF values received for the patient population. Rather, producing the RF-based thoracic fluid information for the patient population may include processing the raw RF values received for the patient population. For example, the computing device may filter the RF values for measurement validity. As an illustration, in addition to receiving RF values for the patient population, the computing device may receive motion information for the patient population (e.g., from accelerometers in the ambulatory medical devices 100 worn by the patient population). The computing device may thus remove any RF values measured when the individual wearing the ambulatory medical device 100 showed a body posture or orientation outside of a predetermined target orientation (e.g., a predetermined percentage outside of a 0 degree and/or 90 degree orientation) and/or a maximal acceleration value larger than a predetermined amount (e.g., larger than 30 milli-g).

As another example, the computing device may receive a calibration vector along with an RF measurement matrix for each individual in the patient population. As such, the computing device may transform the RF measurement matrix according to the calibration vector. An illustration of an equation for calibrating the RF measurement matrix is provided below, where R represents the RF measurement matrix received for a patient, CALIB represents the calibration vector received for the patient, and C represents the calibrated RF measurement matrix:

$$C = \frac{R}{CALIB}$$

In another example, the calibration vector may be simplified to one or more scalar quantities, for instance gain and offset in order to provide a linear calibration. The scalar quantities may also represent a polynomial, exponential, spline, or other nonlinear calibration function, or combinations thereof. As another example, the computing device may perform one or more additional transformations to the calibrated RF measurement matrix or to a raw RF measurement matrix, such as transforming the RF measurement matrix from the frequency domain to the time domain (or vice versa) and/or by averaging one or more measurements in the RF measurement matrix. As another example, the computing device may filter out values from the RF measurement matrix that correspond to ranges that are likely to produce the most useful data (e.g., the data with fewer artifacts and/or the most likely to include information about the relevant depth into the patient's tissue of interest).

As another example, the computing device may, for each individual of the patient population, index the individual's raw or preliminarily transformed RF values (e.g., preliminarily transformed or processed to calibrate the RF values, to remove RF values taken while the patient was moving too much, etc.) by normalizing the raw or transformed RF values to a baseline. As an illustration, the baseline may be an RF value or an average of RF values (e.g., output using the process described above) for the individual taken during an initial baselining operation when the individual was provided with an ambulatory medical device 100. As another illustration, the baseline may be an RF value that correlates with a certain amount of thoracic fluid (e.g., for a patient with the individual's height, weight, BMI, chest circumference, thoracic tissue thickness, and/or the like). For instance, the baseline may be determined by averaging a preconfigured number of impedance measurements, where each measurement in this averaged set is taken at a different time t:

$$baseline = 〈 F ( t ) 〉$$

The computing device may then index the individual's transformed RF values according to, for instance, the following equation, where TI(t) represents the indexed RF values over time:

$$TI ( t ) = \frac{F ( t )}{baseline}$$

As another example, the computing device may, for each individual of the patient population, transform the individual's raw or preliminarily transformed or processed RF values using an alpha function, where the alpha function includes an exponential function based on a rate of attenuation in the received RF waves compared to the transmitted RF waves used to produce the RF values. To illustrate, the computing device may transform the individual's raw or preliminarily transformed or processed RF values using the following equation, where I represents the received RF signals (e.g., the RF waves scattered or reflected from the patient's thoracic region), I₀ represents the initial intensity of the transmitted signal (e.g., the RF waves transmitted by the at least one RF antenna and RF circuitry), α represents a factor of amplitude attenuation (e.g., the attenuation coefficient), and z represents the depth in the tissue penetrated by the RF waves:

$$I = {I}_{0} {e}^{- α z}$$

As another example, the computing device may, for each individual of the patient population, transform the individual's raw or preliminarily transformed RF values according to another type of function, such as a linear function, a second-order polynomial function, a third-order polynomial function, a fourth-order polynomial function, an nth-order polynomial function, a logarithmic function, an exponential function, a power function, and/or the like. Accordingly, the output of any of the above examples, as well as other examples of processing raw RF values, may be the RF-based thoracic fluid information used in the RF-based thoracic fluid model.

In some implementations, the RF-based thoracic fluid model may include at least one type of patient health information as an input, in addition to the RF-based thoracic fluid information. As an example, the RF-based thoracic fluid model may include an individual's BMI and RF-based thoracic fluid information as inputs to the RF-based thoracic fluid model. As another example, the RF-based thoracic fluid model may include the individual's BMI, respiration rate, and RF-based thoracic fluid information as inputs to the RF-based thoracic fluid model. Accordingly, deriving the at least one weighting parameter by optimizing the correlation with the reference thoracic fluid information for the patient population may further include optimizing the correlation between the reference thoracic fluid information, and the RF-based thoracic fluid information and selected patient health information for the patient population. In some implementations, the RF-based thoracic fluid model may include the RF-based thoracic fluid information as multiple inputs to the RF-based thoracic fluid model (e.g., in addition to at least one type of patient health information). For instance, the RF-based thoracic fluid model may include a metric of the RF values indexed to a baseline as a first input and a metric of the RF values transformed by the alpha function as a second input.

Returning back to FIG. 4, once the at least one weighting parameter is retrieved at step 412, the remote server 102 applies the at least one weighting parameter to the at least one RF value for the new patient wearing the ambulatory medical device 100 at step 414. Accordingly, in some implementations, applying the at least one weighting parameter to the at least one RF value for the patient includes transforming the at least one RF value to match the format of the inputs used in the RF-based thoracic fluid model used to produce the at least one weighting parameter, as discussed above. As an illustration, the at least one RF value may be processed, similar to the processing of the RF values for the patient population described above with respect to FIG. 5. For example, the remote server 102 may transform the at least one RF value from a frequency domain to a time domain, or vice versa (e.g., using a fast Fourier transform operation). As another example, the remote server 102 may index the patient's raw or preliminarily transformed at least one RF value by normalizing the raw or preliminarily transformed at least one RF value to a baseline. As another example, the remote server 102 may transform the raw or preliminarily transformed at least one RF value using the alpha function described above. As another example, the remote server 102 may transform the patient's raw or preliminarily transformed at least one RF value according to another type of function, such as a linear function, a second-order polynomial function, a third-order polynomial function, a fourth-order polynomial function, an nth-order polynomial function, a logarithmic function, an exponential function, a power function, and/or the like. At least one weighting parameter may be applied to the output of any of the above examples, as well as other examples of processing the patient's at least one RF value, as part of step 414.

In some implementations, applying the at least one weighting parameter further includes applying the at least one weighting parameter to the RF value and to at least one patient health value to determine the amount of thoracic fluid in the patient. As such, the remote server 102 may identify at least one patient health value for the new patient wearing the ambulatory medical device 100. Similar to the patient health information described with respect to step 506 of FIG. 5, the at least one patient health value may include at least one demographic for the patient, at least one biometric for the patient, and/or at least one anthropomorphic parameter for the patient. For example, the at least one patient health value may include the gender, age, height, weight, BMI, heart rate, respiration rate, heart failure type, comorbidity or comorbidities, cardiac event or cardiac events, chest circumference, thoracic tissue thickness, activity level, posture, and/or the like for the patient.

In some implementations, identifying the at least one patient health value for the patient may include receiving at least one patient health value as a user input, such as an input from the patient's caregiver (e.g., via a caregiver interface 118). In some implementations, the remote server 102 may identify at least one patient health value from signals including health information for the patient. For example, the remote server 102 may receive ECG signals from the ambulatory medical device 100 (e.g., recorded by the ECG electrodes 114). As such, the remote server 102 may identify the at least one patient health value from the received ECG signals, such as by determining a heart rate or cardiac events for the patient from the received ECG signals. As another example, the remote server 102 may receive motion signals associated with the patient from the ambulatory medical device 100 (e.g., recorded by the accelerometer 1022). The remote server 102 may identify the at least one patient health value from the motion signals, such as by determining a respiration rate, an activity level, or posture of the patient from the motion signals. In some implementations, the at least one patient health value may be a statistical measure (e.g., average, median, maximum, minimum, mode, etc.) of a series of patient health values, such as an average or median heart rate or respiration rate taken over a predetermined period of time.

In some implementations, applying the at least one weighting parameter to determine the amount of thoracic fluid for the patient may include calibrating the at least one weighting parameter to the patient. In some examples, calibration may be accomplished by including at least one patient health value in the determination of the amount of thoracic fluid. To illustrate, calibrating the at least one weighting parameter to the patient may occur when the RF-based thoracic fluid model includes BMI information, chest circumference information, thoracic tissue thickness information, and/or the like. Thus, applying the at least one weighting parameter to determine the amount of thoracic fluid for the patient includes applying the at least one weighting parameter to a BMI, chest circumference, thoracic tissue thickness, and/or the like for the patient, in addition to the at least one RF value. In this sense, these types of patient health values may be considered calibration values.

In some examples, the remote server 102 may identify at least one calibration value for the patient. The at least one calibration value may be based on demographic, biometric, anthropomorphic, or other physiological data for the patient. As an example, the remote server 102 may receive at least one CT scan value for the patient, where the at least one CT scan value for the patient is based on a reference CT scan for the patient. For instance, the remote server 102 may receive a calibration CT scan for the patient and determine the percent of thoracic fluid to thoracic tissue of interest (e.g., similar to the process used in step 502 of FIG. 5), or the remote server 102 may receive calibration CT scan data from a calibration CT scan taken for the patient where the calibration CT scan data indicates the percent of thoracic fluid to thoracic tissue of interest. As another example, the remote server 102 may use a function or algorithm that produces a calibration value based on, for instance, the patient's age, gender, height, weight, BMI, percent body fat, thoracic tissue thickness, chest circumference, and/or the like (which the remote server 102 may receive as user inputs from a caregiver of the patient). As another example, the remote server 102 may determine at least one calibration value for the patient by determining a rate of attenuation in received RF waves (e.g., RF waves transmitted through the patient's thoracic region and/or reflected from the patient's thoracic region) compared to transmitted RF waves (e.g., the RF waves transmitted by the at least one RF antenna of the ambulatory medical device 100). The remote server 102 may then determine at least one calibration value for the patient based on the amount of attenuation in the received RF waves compared to the transmitted RF waves. In some examples, other biometric information may be used to determine at least one calibration value, such as an ECG, x-ray information, blood test results, and/or the like.

The remote server 102 may then use the at least one calibration value to calibrate the application of the at least one weighting parameter to the patient. In some implementations, applying the at least one weighting parameter to the at least one RF value (and, in some examples, to the at least one patient value) may also include applying the at least one weighting parameter to the at least one calibration value. For example, a calibration value may be multiplied by one of the weighting parameters (e.g., with the RF-based thoracic fluid model used to produce the at least one weighting parameter using a similar set of calibration values for the patient population). In some implementations, applying the at least one weighting parameter to the at least one RF value (and, in some examples, to the at least one patient value) may produce a preliminary amount of thoracic fluid in the patient. The remote server 102 may then adjust the preliminary amount of thoracic fluid in the patient using the at least one calibration value to produce the final amount of thoracic fluid in the patient. As an example, after the at least one weighting parameter has been applied, the at least one calibration value may be added to, subtracted from, multiplied by, etc. the preliminary result. As another example, the preliminary amount of thoracic fluid in the patient may be input into a calibration equation (e.g., produced based on health data about the patient, such as the patient's gender, age, height, weight, chest circumference, etc.), and the output of the calibration equation may be the final amount of thoracic fluid. In some implementations, the remote server 102 may use the at least one calibration value to calibrate the application of the at least one weighting parameter to the patient using a combination of the above examples.

In some implementations, the remote server 102 may alternatively or additionally calibrate for noise or artifacts that may have been introduced into the at least one RF value for the patient (e.g., based on “intrasubject” factors). As an example, the remote server 102 may receive information on where the patch was placed on the patient (e.g., as a user input from the patient or a caregiver) and calibrate the at least one RF value to account for the patch placement. As another example, the remote server 102 may receive motion data for the patient (e.g., from a motion sensor on the ambulatory medical device 100) and calibrate the at least one RF value to account for movement of the patient while RF measurements were being taken (e.g., to remove motion artifacts). As another example, the remote server 102 may receive a time at which the RF measurements were taken and adjust the at least one RF value to account for circadian changes in thoracic fluid. These intrasubject calibrations or adjustments may be based on empirical data gathered from a patient population, machine-learning techniques, and so on.

Once the remote server 102 has applied the at least one weighting parameter to the at least one RF value (and, in some implementations, to the at least one patient value and/or at least one calibration value), the remote server 102 outputs the determined amount of thoracic fluid at step 416. The output amount of thoracic fluid may be as a percentage (e.g., a percentage of lung fluid to lung tissue), as an absolute amount of fluid, and so on. Additionally, depending on, for example, the RF-based thoracic fluid model used, the output amount of thoracic fluid may be correlated with the patient's extravascular thoracic fluid (e.g., extravascular lung fluid) or intravascular and extravascular thoracic fluid (e.g., intravascular and extravascular lung fluid), as described above with respect to FIG. 5. In some examples, the output amount of thoracic fluid may be further classified into buckets. For example, an output amount of thoracic fluid between a first predetermined value and a second predetermined value may be classified into a first bucket, an output amount of thoracic fluid between the second predetermined value and a third predetermined value may be classified into a second bucket, and so on. These buckets may further have assigned descriptors. To illustrate, a first bucket may be a “low thoracic fluid” bucket, a second bucket may be a “normal thoracic fluid” bucket, a third bucket may be a “high thoracic fluid” bucket, a fourth bucket may be a “very high thoracic fluid” bucket, and so on. As another illustration, a first bucket may be a “normal” bucket (e.g., less than or equal to 15% lung fluid), a second bucket may be a “possibly fluid overloaded” bucket (e.g., between 15% lung fluid and 20% lung fluid), and a third bucket may be a “fluid overloaded” bucket (e.g., greater than or equal to 20% lung fluid).

In some implementations, the remote server 102 may store the amount of thoracic fluid for later evaluation or processing, such as for use in a patient report prepared by the remote server 102 and/or technician interface 116 and provided to a caregiver interface 118 as described above. In some implementations, the remote server 102 may periodically determine amounts of thoracic fluid, which may be used to show the patient's amount of thoracic fluid over time (e.g., in a report prepared for a caregiver and/or patient). In some implementations, the remote server 102 may determine a starting baseline amount of thoracic fluid using the above processes and subsequently determine a relative amount of thoracic fluid over time. In this way, the remote server 102 may use the relative amounts of thoracic fluid to show whether the patient's thoracic fluid is trending upward, downward, or the same compared to the baseline amount of thoracic fluid. In some implementations, the remote server 102 may issue an alert to the patient's caregiver if, for example, the determined amount of thoracic fluid transgresses one or more predetermined alert thresholds. The urgency of the alert may depend on which predetermined alert threshold the determined amount of thoracic fluid is above. For instance, if the determined amount of thoracic fluid is above a first threshold, the remote server 102 may send an alert to the caregiver (e.g., via the caregiver interface 118), informing the caregiver of the amount of thoracic fluid determined for the patient. If the determined amount of thoracic fluid is above a second, higher threshold, the remote server 102 may call the caregiver to inform the caregiver of the amount of thoracic fluid determined for the patient.

Although the sample process 400 of FIG. 4 is described as occurring partially at the ambulatory medical device 100 and partially at the remote server 102, in some implementations, the sample process 400 of FIG. 4 may occur entirely on the ambulatory medical device 100. For example, steps 410-416 of FIG. 4 may be carried out by the monitoring unit 104 and/or the portable gateway 108 of the ambulatory medical device 100, or by any other processing unit described in any of the embodiments of the ambulatory medical device 100 below. In such embodiments, the ambulatory medical device 100 may transmit the output amount of thoracic fluid to the remote server 102 (e.g., to be included in a report prepared for a caregiver) and/or directly to a caregiver interface 118.

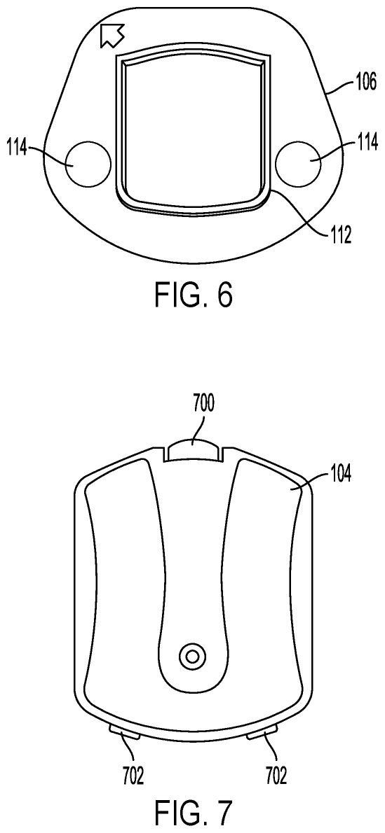

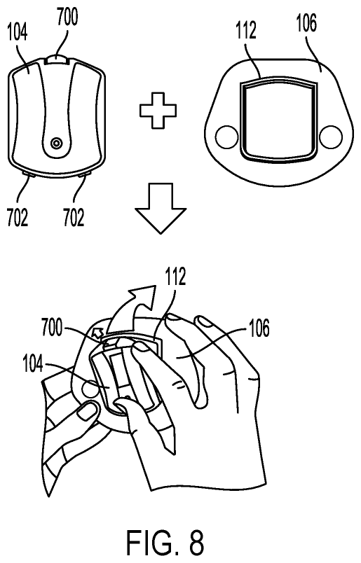

Returning to the monitoring unit 104 and the adhesive patch 106 from the embodiment of the ambulatory medical device 100 shown in FIG. 1, FIGS. 6-8 show the monitoring unit 104 and the adhesive patch 106 according to some implementations. The adhesive patch 106 may be disposable (e.g., single-or few-use patches) and may be made of a biocompatible, non-woven material. Additionally, as shown in FIG. 6, and as noted above, the adhesive patch 106 may include a patch frame 112 delineating the boundary of the region of the patch 106 that is configured to house the monitoring unit 104. In some embodiments, the monitoring unit 104 may be designed for long-term usage. In such embodiments, the connection between the adhesive patch 106 and the monitoring unit 104 may be configured to be reversible, e.g., the monitoring unit 104 may be configured to be removably attached to the adhesive patch 106. For example, as shown in FIG. 7, the monitoring unit 104 may include components such as one or more snap-in clips 700 that are configured to secure the monitoring unit 104 to the adhesive patch 106 upon attachment to the patch frame 112. After the monitoring unit 104 is attached to the patch frame 112, for instance, a user may press the snap-in clip 700 to subsequently release the monitoring unit 104 from the patch frame 112. In some implementations, the monitoring unit 104 may also include positioning tabs 702 that facilitate the attachment process between the monitoring unit 104 and the adhesive patch 106. For example, the positioning tabs 702 may guide a user to insert the monitoring unit 104 onto the correct portion of the patch frame 112 such that the monitoring unit can then be coupled, connected, or snapped into the patch frame 112 using the snap-in clip 700, as shown in FIG. 8.

In some implementations, the adhesive patch 106 may be designed to maintain attachment to skin of a patient for several days (e.g., in a range from about 4 days to about 10 days, from about 3 days to about 5 days, from about 5 days to about 7 days, from about 7 days to about 10 days, from about 10 days to about 14 days, from about 14 days to about 30 days, etc.). After the period of use, the adhesive patch 106 can be removed from the patient's skin and the monitoring unit 104 can be removed from the patch 106. The monitoring unit 104 can be removably coupled, connected, or snapped onto a new adhesive patch 106 and reapplied to the patient's skin.

In some implementations, the adhesive patch 106 may include additional components that facilitate or aid with the monitoring and/or recording or acquiring of additional physiological data by the monitoring unit 104. For instance, as discussed above, the adhesive patch 106 may include conductive elements such as one or more ECG electrodes 114 (e.g., a single lead, two leads, etc.) that can be used when recording ECG signals from the surface (e.g., skin contacted directly or through a covering) of a patient's body. The electrodes 114 may be coupled to the monitoring unit 104 by dedicated wiring within the patch 106. In some implementations, the ECG may have a sampling rate in the range from about 250 Hz to about 500 Hz, from about 300 Hz to about 450 Hz, or from about 350 Hz to about 400 Hz, including values and subranges therebetween. In some embodiments, the ECG signal may be sampled after band-pass filtering by a 12-bit analog-to-digital converter (“ADC”). During normal operation, data may be transferred to the remote server 102 “as-is” and can then be used by the remote server 102 for analysis. In some implementations, an internal process allows for real-time evaluation of the ECG signal quality upon each attachment of the ambulatory medical device 100 to the patient. For example, the monitoring unit 104, portable gateway 108, and/or remote server 102 may evaluate a signal quality by analyzing the fidelity of an ECG waveform recorded by the monitoring unit 104. As another example, the monitoring unit 104 and/or adhesive patch 106 may transmit a testing signal to the patient's body that may be picked up by the ECG electrodes 114 to determine whether the ECG electrodes 114 are properly adhered to the patient's skin and monitoring the patient's ECG signals.

In some implementations, the remote server 102 and/or the ambulatory medical device 100 (e.g., at the monitoring unit 104 and/or portable gateway 108) may process the ECG signals to detect an arrhythmia of the patient. Types of arrhythmias detected by the remote server 102 and/or the ambulatory medical device 100 may include ventricular ectopic beats (VEB), ventricular runs/ventricular tachycardia, bigeminy, supraventricular ectopic beats (SVEB), supraventricular tachycardia, atrial fibrillation, ventricular fibrillation, pauses, 2nd AV blocks, 3rd AV blocks, bradycardia, and/or other types of tachycardia. Additionally, in some implementations, the remote server 102 and/or ambulatory medical device 100 may perform other processing or analyses of the ECG signal, such as band pass filtering, detecting R-R intervals, detecting QRS intervals, and/or heart rate estimation.

Aside from ECG electrodes 114, the ambulatory medical device 100 may include other types of additional physiological sensors, in some implementations. These additional physiological sensors may be implemented, for example, at the monitoring unit 104, the adhesive patch 106, and/or the portable gateway 108. For example, the ambulatory medical device 100 may include a motion sensor (e.g., which may sense changes in a patient's motion and/or posture or position, and may in turn be used to determine the patient's posture, activity level, and respiration rate), a P-wave sensor (e.g., a sensor configured to monitor and isolate P-waves within an ECG waveform), a temperature sensor, an oxygen saturation sensor (e.g., implemented through photoplethysmography, such as through light sources and light sensors configured to transmit light into the patient's body and receive transmitted and/or reflected light containing information about the patient's oxygen saturation), a heart sounds or acoustics sensor, a heart vibrations sensor, and so on.

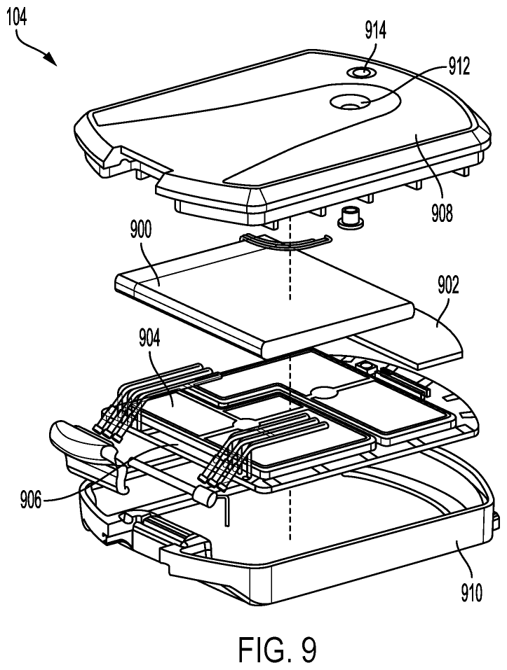

FIG. 9 provides an exploded view of the monitoring unit 104, according to some implementations. The exploded view of FIG. 9 illustrates various components of the monitoring unit 104. For example, the monitoring unit 104 may include a power source, such as a battery 900. In examples, the battery 900 may be a rechargeable lithium ion battery configured to supply power for at least one month of continuous or near-continuous RF recording and/or recording of other physiological signals, such as ECG signals, from the patient. The monitoring unit 104 may also include a wireless communications circuit 902; a radiofrequency shield 904 (e.g., a metallic cover, for instance, to prevent interference with the RF processing and other digital circuitry); a digital circuit board 906; and/or the like. The wireless communications circuit 902 may be a Bluetooth® unit, in some implementations, although in addition to or alternatively to the Bluetooth® unit, other modules facilitating other types of communications (e.g., Wi-Fi, cellular, etc.) may be included in the monitoring unit 104.

In some implementations, the monitoring unit 104 may be configured to monitor, record, and transmit signals (e.g., signals including the at least one RF value, signals including ECG information) to the portable gateway 108 continuously (e.g., via the wireless communications circuit 902). The monitoring unit 104 monitoring and/or recording additional data may not interrupt the transmission of already acquired data to the portable gateway 108. As such, in some implementations, both the monitoring/recording and the transmission processes may occur at the same time or nearly the same time. In some implementations, if the monitoring unit 104 does suspend the monitoring and/or recording of additional data while the monitoring unit 104 is transmitting already acquired data to the portable gateway 108, the monitoring unit 104 may then resume monitoring and/or recording of additional data before all of the already-acquired data has been transmitted to the portable gateway 108. To illustrate, the interruption period for the monitoring and/or recording of additional data may be less in comparison to the time it takes the monitoring unit 104 to transmit the already-acquired data (e.g., the interruption period being between about 0% to 80%, about 0% to about 60%, about 0% to about 40%, about 0% to about 20%, about 0% to about 10%, about 0% to about 5%, including values and subranges therebetween, of the monitoring and/or recording period). This moderate interruption period may facilitate the near-continuous monitoring and/or recording of additional data during transmission of already-acquired physiological data. For example, in one scenario, when a measurement time is about two minutes, any period of suspension or interruption in the monitoring and/or recording of subsequent measurement data may range from a few milliseconds to about a minute. Illustrative reasons for such suspension or interruption of data may include allowing for the completion of certain data integrity and/or other online tests of previously acquired data. In some implementations, if the previous data have problems, the monitoring unit 104 may notify the patient and/or a remote technician of the problems so that appropriate adjustments can be made.

In some implementations, the monitoring unit 104 may be configured to monitor, record, and transmit some data in a continuous or near-continuous manner, as discussed above, while monitoring, recording, and transmitting some other data in a non-continuous manner (e.g., periodically, non-periodically, etc.). For example, the monitoring unit 104 may be configured to record and transmit ECG data from the ECG electrodes 114 continuously or nearly continuously while data from the at least one RF antenna and RF circuitry is transmitted periodically (e.g., because RF measurements may be taken only when the patient is in a good position for transmitting and receiving RF waves, such as when the patient is not moving). As an illustration, ECG data may be transmitted to the portable gateway 108 (and, via the portable gateway 108, to the remote server 102) continuously or near-continuously as additional ECG data is being recorded, while the at least one RF value generated by the at least one RF antenna and RF circuitry may be transmitted once the RF measuring process is completed. In some implementations, monitoring and/or recording of signals by the monitoring unit 104 may be periodic and, in some implementations, may be accomplished as scheduled (e.g., periodically) without delay or latency during the transmission of already acquired data to the portable gateway 108. For example, the monitoring unit 104 may take measurements from the patient and transmit the data generated from the measurements to the portable gateway 108 in a continuous manner as described above.

Additionally, in some implementations, the portable gateway 108 may continuously transmit the signals provided by the monitoring unit 104 to the remote server 102. Thus, for example, the portable gateway 108 may transmit the signals from the monitoring unit 104 to the remote server 102 with little or no delay or latency. To this end, in the context of data transmission between the ambulatory medical device 100 and the remote server 102, continuously includes continuous (e.g., without interruption) or near continuous (e.g., within one minute after completion of a measurement and/or an occurrence of an event on the monitoring unit 104). Continuity may also be achieved by repetitive successive bursts of transmission (e.g., high-speed transmission). Similarly, immediate data transmission may include data transmission occurring or done immediately or nearly immediately (e.g., within one minute after the completion of a measurement and/or an occurrence of an event on the monitoring unit 104).

Further, in the context of signal acquisition and transmission by the ambulatory medical device 100, continuously may also include uninterrupted collection of data sensed by the cardiovascular monitoring unit 104, such as RF measurements that produce the at least one RF value, with clinical continuity. In this case, short interruptions in data acquisition of up to one second several times an hour, or longer interruptions of a few minutes several times a day, may be tolerated and still seen as continuous. As to latency as a result of such a continuous scheme as described herein, the overall amount of response time (e.g., time from when an event onset is detected to when a notification regarding the event is issued) can amount, for example, from about five to fifteen minutes. As such, transmission/acquisition latency may therefore be in the order of minutes.

In some implementations, the bandwidth of the link between the monitoring unit 104 and the portable gateway 108 may be larger, and in some instances, significantly larger, than the bandwidth of the acquired data to be transmitted via the link (e.g., burst transmissions). Such implementations may ameliorate issues that may arise during link interruptions, periods of reduced/absent reception, etc. In some implementations, when transmission is resumed after an interruption, the resumption may be in the form of last-in-first-out (LIFO). In some implementations, the portable gateway 108 additionally may be configured to operate in a store and forward mode, where the data received from the monitoring unit 104 is first stored in an onboard memory of the portable gateway 108 and then forwarded to the remote server 102. In some implementations, the portable gateway 108 may function as a pipeline and pass through data from the monitoring unit 104 immediately to the remote server 102. Further, in some implementations, the data from the monitoring unit 104 may be compressed using data compression techniques to reduce memory requirements as well as transmission times and power consumption. In some implementations, the link between the portable gateway 108 and the remote server 102 may similarly be larger, and in some instances, significantly larger, than the bandwidth of the data to be transmitted via the portable gateway 108 and the remote server 102.

Referring back to FIG. 9, the components of the monitoring unit 104 (e.g., the battery 900, communications circuit 902, radiofrequency shield 904, digital circuit board 906, and/or the like) may be provided between a front cover 908 forming an upper surface of the monitoring unit 104 and a back cover 910 forming a bottom surface of the monitoring unit 104. For instance, the back cover 910 may be configured to contact the adhesive patch 106, and the front cover 908 may be configured to face away from the patient such that the front cover 908 is accessible when the monitoring unit 104 is attached to the adhesive patch 106. In some implementations, an indicator light 912 and/or a button 914 may be embedded into the front cover 908 visible through the upper surface. The indicator light 912 may provide feedback on the status of the monitoring unit 104 and its components, such as the charging and/or power level of the power source of the monitoring unit 104 (e.g., the battery 900), the attachment level of the monitoring unit 104 to the adhesive patch 106, the attachment level of the adhesive patch 106 to the surface of the body to which the adhesive patch 106 is attached, etc.

The button 914 may be configured for the patient and/or caregiver to provide feedback to the monitoring unit 104 and/or, via the monitoring unit 104, to the remote server 102. For instance, the button 914 may allow the patient and/or caregiver to activate or deactivate the monitoring unit 104. In some implementations, the button 914 may be used to reset the monitoring unit 104, as well as pair the monitoring unit 104 to the portable gateway 108 and initiate communication with the portable gateway 108. In some implementations, the button 914 may allow a user to set the monitoring unit 104 in an “airplane mode,” for example, by deactivating any wireless communication (e.g., Wi-Fi, Bluetooth®, etc.) with external devices and/or servers, such as the portable gateway 108 and/or the remote server 102.

FIG. 10 illustrates an example electronic architecture for the monitoring unit 104. In some implementations, as shown in FIG. 10, the monitoring unit 104 includes one or more external interfaces, either connected to or embedded in the monitoring unit 104. For example, the monitoring unit 104 may include the button or switch 914 for activating the monitoring unit 104, deactivating the monitoring unit 104, pairing the monitoring unit 104 with the portable gateway 108, receiving patient input, and/or the like. In some implementations, the monitoring unit 104 may also include the indicator light 912 and a buzzer 1000 for providing light and/or audio feedback to a user of the monitoring unit 104 (e.g., in response to the patient activating the button 914 or tapping the monitoring unit 104 to record that the patient is experiencing symptoms suspected to be related to an arrhythmia).

Further, in some embodiments, the monitoring unit 104 may be connectable to the ECG pads or electrodes 114 coupled to the patient (e.g., connectable to the ECG pads 114 embedded in the adhesive patch 106) and to a charger, such as charger 110, via charging link 1002. For instance, the back cover 910 of the monitoring unit 104 may include metal contacts configured to connect to the ECG pads 114 when the monitoring unit 104 is attached to the adhesive patch 106 and to a charging power source when the monitoring unit 104 is attached to the charger 110. The ECG circuits 1020 may receive signals from the ECG pads 114 when the monitoring unit 104 is attached to the adhesive patch 106, where the signals received from the ECG pads 114 include ECG waveforms sensed from the patient. Alternatively, or additionally, in some implementations, the monitoring unit 104 may include an inductive circuit configured to charge the monitoring unit 104 via a wireless inductive charging link 1002. As shown in FIG. 10, the charging link 1002 may be coupled to a power management circuit 1004 (e.g., when the monitoring unit 104 is attached to the charger 110, when the monitoring unit 104 is placed in proximity to an inductive charging pad), where the power management circuit 1004 is configured to charge an onboard power source such as the battery 900.

Internally, in some implementations, the monitoring unit 104 may include a microprocessor (e.g., being connected to a separate non-volatile memory, such as memory 1008) or a microcontroller 1006. The microcontroller 1006 stores instructions specifying how measurements (e.g., RF, ECG, accelerometer, etc.) are taken, how obtained data are transmitted, how to relay a status of the monitoring unit 104, how/when the monitoring unit 104 can enter a sleep level, and/or the like. In some implementations, the instructions may also specify the conditions for performing certain types of measurements. For example, the instructions may specify that a sensor of the monitoring unit 104 may not commence measurements (e.g., RF measurements, ECG measurements, respiration rate measurements, etc.) unless the patient using the monitoring unit 104 is at rest or maintaining a certain posture (e.g., as indicated by data from an accelerometer 1022 of the monitoring unit 104). As another example, the instructions may identify the conditions that may have to be fulfilled before measurements can commence, such as a sufficient attachment level between the monitoring unit 104 and the adhesive patch 106 and/or a sufficient attachment level between the adhesive patch 106 and the surface of the body onto which the adhesive patch 106 is attached. In some implementations, the microcontroller 1006 may have internal and/or external non-volatile memory banks (e.g., memory 1008) that can be used for measurement directories and data, scheduler information, and/or a log of actions and errors. This non-volatile memory may allow for retaining data and status information in the case of a total power down.

As discussed above, in various implementations, the monitoring unit 104 includes at least one RF antenna and RF circuitry for transmitting electromagnetic waves into the body of a patient (e.g., into a thoracic region of the patient) and receiving electromagnetic waves that are transmitted through the patient's body and/or scattered or reflected from internal tissues of the patient's body. For example, the at least one RF antenna and RF circuitry may transmit RF waves in a range from about 0.1 GHz to about 5 GHz (e.g., including a small amount above and below this range, such as 10% less than 0.1 GHz and 10% more than 5GHz, 5% less than 0.1 GHz and 5% more than 5GHz, and so on). The at least one RF antenna may be flat, printed, set flush against the skin, with or without an interface material, and/or the like. The at least one RF antenna may be in a bow-tie, spiral, monostatic, bistatic, and or like configurations. Further, the RF circuitry is configured to process the received electromagnetic waves so as to determine some properties of the tissues that are on the path of the transmitted and/or received electromagnetic waves. The RF circuitry may process the received electromagnetic waves to produce at least one RF value that the monitoring unit 104 may transmit to the remote server 102 and/or analyze to determine, for instance, an amount of fluid in the patient's thoracic region. To illustrate, the at least one RF antenna and RF circuitry may transmit electromagnetic waves towards a thoracic region of a patient that includes the patient's lungs and receive electromagnetic waves transmitted through and/or scattered or reflected by the patient's lung tissues. As such, the received electrometric waves may include information about a level of fluid in or near the patient's lungs, which may be contained by the at least one RF value produced by the RF circuitry of the monitoring unit 104.

In some implementations, the at least one RF antenna and RF circuitry are configured to transmit a low-power signal in an ultra-high frequency band (e.g., 0.1 GHz to 5.0 GHz, 0.5 GHz to 2.1 GHZ) at a predetermined rate (e.g., every 10 ms, every 20 ms, every 30 ms, every 40 ms, every 50 ms, etc.). The RF antenna and RF circuitry transmit a burst of a predetermined number of frequencies (e.g., 32 frequencies, 64 frequencies, 128 frequencies) spanning the ultra-high frequency band at the predetermined rate. The at least one RF antenna and RF circuitry receive RF waves transmitted through the patient and/or scattered or reflected from the patient for a predetermined amount of time (e.g., about 30 seconds, about 1 minute, about 2 minutes, about 3 minutes, about 5 minutes, about 10 minutes, etc.). In some implementations, the ambulatory medical device 100 (e.g., at the monitoring unit 104 and/or the portable gateway 108) and/or the remote server 102 are configured to gate when RF measurements are taken and/or discard certain RF measurements based on the patient's state when the RF measurements were taken. For example, the ambulatory medical device 100 and/or the remote server 102 may determine whether the patient showed movement above a predetermined threshold while the RF sampling was taking place, as discussed above with respect to step 404 of FIG. 4.

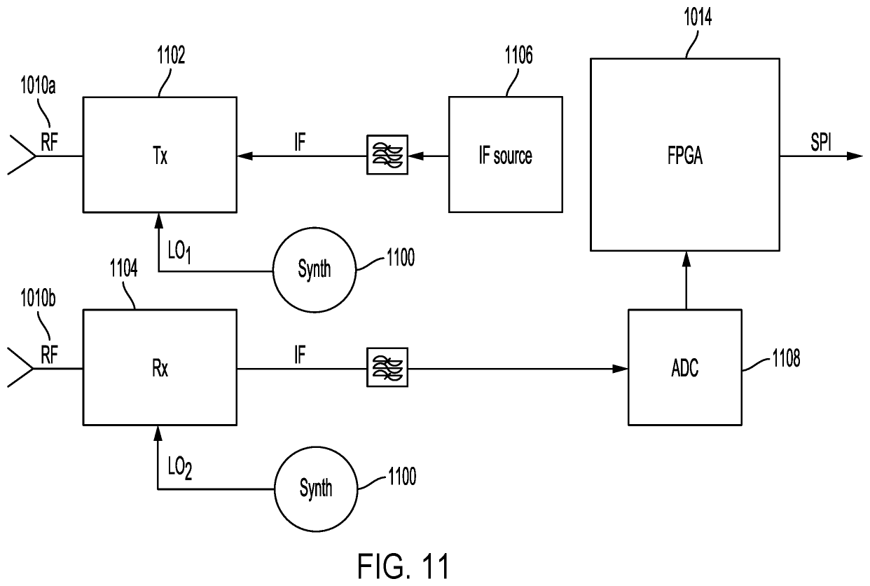

Accordingly, FIG. 10 shows an example embodiment of the monitoring unit 104 including RF antennae 1010a, 1010b, an RF circuit 1012, and other circuits for controlling the RF circuit 1012 (e.g., field-programmable gate array (FPGA) circuits 1014). In various implementations, the RF antennae 1010a, 1010b are configured to transmit RF waves into the body of a patient to which the monitoring unit 104 is attached and receive transmitted and/or scattered or reflected RF waves from the body of the patient. FIG. 11 shows block diagrams illustrating examples of RF sensor functionality implemented within the RF circuit 1012. Such functionality may be used to monitor, for example, the amount of fluid in the patient's thoracic region, either statically or over time, in accordance with the techniques described herein.

As shown in FIG. 11, initially, one or more RF signals (e.g., a single local oscillator (LO) signal, or different “LO₁” and “LO₂” signals, collectively “LO signals”) can be generated by a broadband synthesizer 1100 (e.g., a pulse generator and synthesizer, or local oscillator). Such a synthesizer 1100 may include moderate phase noise performance and/or fast settling time capabilities. According to the embodiment shown in FIG. 11, the RF circuit 1012 also includes a transmitter portion 1102 (e.g., Tx) coupled to a transmitter RF antenna, such as antenna 1010a, and associated circuitry for transmitting RF waves, for example, to the patient's thoracic region. According to FIG. 11, the RF circuit 1012 further includes a receiver portion 1104 (e.g., Rx) coupled to a receiver antenna, such as antenna 1010b, and associated circuitry for receiving transmitted and/or reflected or scattered RF waves from, for example, the patient's thoracic region. Alternatively, in some implementations, the RF sensor may be implemented with one RF antenna or with more than two RF antennae.

In some implementations, the LO signal of the transmitter portion 1102 is multiplied with an external sine wave at a low frequency intermediate frequency (IF) signal, generated by an IF source 1106, and directed to the output of the transmitter portion 1102. the LO signal at the transmitter portion 1102 and the receiver portion 1104 can be generated by one or more LO sources (e.g., synthesizer(s) 1100). Output power may be controlled via a digitally controlled attenuator (DCA) on the LO signal transmitter path. An external, reflected RF wave returning to the receiver RF antenna may be directed to the receiver portion 1104 and down-converted to an IF frequency by a down conversion mixer. The reflection characteristics (e.g., phase and amplitude) can be transformed to a new IF carrier (e.g., on the order of 250 kHz), filtered, and amplified, before being forwarded to an analog-to-digital converter (ADC) 1108. In some implementations, digital control for the functionality described with respect to FIG. 11 may be achieved directly by a processor and/or digital logic (e.g., an FPGA 1014), which may be configured to control the transmitter and receiver configuration processes, IF signal adjustments, and associated switching. As shown in FIG. 11, the output of the RF circuit 1012 may be in the form of serial peripheral interface (SPI).

Referring back to FIG. 10, as discussed above, the monitoring unit 104 may also include or be connected to one or more additional sensors, according to some implementations. For example, as shown in FIG. 10, the monitoring unit 104 may include a motion sensor such as a 3D accelerometer 1022. Using the 3D accelerometer 1022, the monitoring unit 104 may acquire data on patient movements, patient orientation, patient respiration, and/or the like. The ambulatory medical device 100 (e.g., at the monitoring unit 104 and/or portable gateway 108) and/or the remote server 102 may use the acquired accelerometer data to determine one or more patient health values, such as the patient's respiration rate, heart rate, activity rate, posture or orientation, and/or the like. In some implementations, the monitoring unit 104 may use the patient health value(s), for example, to determine when to take RF measurements from the patient. As an illustration, to reduce artifacts and other poor readings, the monitoring unit 104 may only or primarily take RF measurements from the patient when the monitoring unit 104 determines from the accelerometer data that the patient is substantially stationary or otherwise inactive. For example, the monitoring unit 104 may determine from the accelerometer data (e.g., accelerometer counts) that the patient motion is below a preset threshold to determine that the patient is substantially stationary or otherwise inactive.

However, in some implementations, the ambulatory medical device 100 may be implemented differently from the configuration shown in FIGS. 1 and 6-9. For example, some implementations of the ambulatory medical device 100 may not include the portable gateway 108, as discussed above. As another example, in some implementations, the ambulatory medical device 100 may not include an adhesive patch 106. Instead, the monitoring unit 104 may be mounted on another mechanical implement configured to be removably attached to the patient's body. For instance, the ambulatory medical device 100 may include a band configured to encircle the patient's chest. The band may be made of an elastic material that compresses the band around the patient's chest to ensure a secure or relatively secure fit where the band does not slip on the patient's chest as the patient moves. The monitoring unit 104 may accordingly be mounted on the band, such as on a plastic frame similar to the frame 112 of the adhesive patch 106 shown in FIGS. 1, 6, and 8, or on mating hooks, strips of hook-and-loop cloth, and/or the like.

As another example, in some implementations, the ambulatory medical device 100 may include more than one monitoring unit 104 and/or may split the functionalities of the monitoring unit 104 between multiple devices. To illustrate, as discussed above, monitoring unit 104 may be in communication with a secondary monitoring unit configured to be placed on the other side of the patient's thoracic region from the monitoring unit 104 to receive RF waves transmitted through the patient's thoracic region from the monitoring unit 104. As another example, in some implementations, the adhesive patch 106 may be worn on one location of the patient's body (e.g., on the patient's side, such as in the position shown in FIG. 2), with the monitoring unit 104 mounted on the adhesive patch 106. The monitoring unit 104 and/or the adhesive patch 106 may then be in a wired or wireless communication with one or more sensors, such as patch ECG electrodes adhered directly to the patient's skin, worn at a second location of the patient's body (e.g., patch ECG electrodes worn on the patient's chest). As another example, in some implementations, the monitoring unit 104 may be worn without the adhesive patch 106 at a first location on or near the patient, such as worn on a belt at the patient's waist. The monitoring unit 104 may then be in a wired or wireless communication with one or more sensors, such as patch ECG electrodes and at least one patch RF antenna adhered directly to the patient's skin, worn at a second location of the patient's body, such as on the patient's chest.

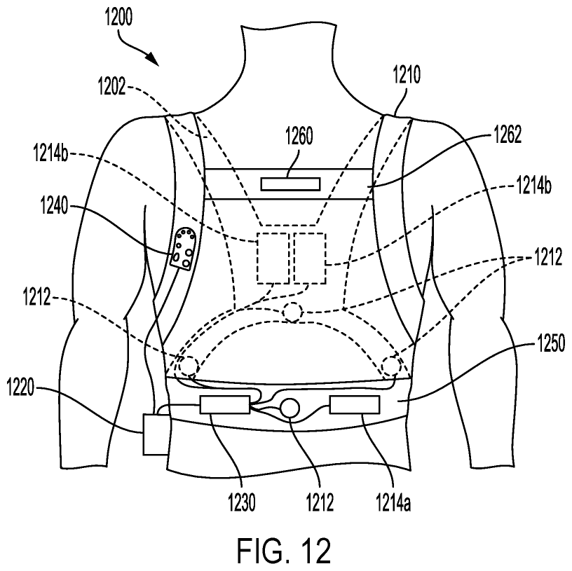

FIG. 12 also shows another embodiment of the ambulatory medical device 100, wherein the ambulatory medical device 100 includes a garment device 1200. The garment device 1200 shown in FIG. 12 is external and wearable by the patient 1202 around the patient's torso. Such a garment device 1200 can be, for example, capable of and designed for moving with the patient 1202 as the patient 1202 goes about their daily routine. For example, the garment device 1200 as described herein can be bodily-attached to the patient 1202, such as the LifeVest® wearable cardioverter defibrillator available from ZOLL® Medical Corporation of Chelmsford, MA. In one example scenario, such wearable defibrillators can be worn nearly continuously or substantially continuously for a week, two weeks, a month, or two or three months at a time. During the period of time in which they are worn by the patient 1202, the wearable defibrillators can be configured to continuously or substantially continuously monitor the vital signs of the patient 1202 and, upon determination that treatment is required, can be configured to deliver one or more therapeutic electrical pulses to the patient 1202. For example, such therapeutic shocks can be pacing, defibrillation, cardioversion, or transcutaneous electrical nerve stimulation (TENS) pulses.

The garment device 1200 can include one or more of the following: a garment 1210 configured to be worn about the patient's torso, one or more sensing electrodes 1212 (e.g., ECG electrodes), one or more therapy electrodes 1214a and 1214b (collectively referred to herein as therapy electrodes 1214), a medical device controller 1220, a connection pod 1230, a patient interface pod 1240, a belt 1250, or any combination of these. In some examples, at least some of the components of the garment device 1200 can be configured to be affixed to the garment 1210, such as by mating hooks, hook-and-loop fabric strips, receptacles (e.g., pockets), and the like. In some examples, at least some of the components of the garment device 1200 can be permanently integrated into the garment 1210, such as by being sewn into the garment. In some examples, at least some of the components may be connected to each other through cables, through sewn-in connections (e.g., wires woven into the fabric of the garment 1210), through conductive fabric of the garment 1210, and/or the like.

The medical device controller 1220 can be operatively coupled to the sensing electrodes 1212, which can be affixed to the garment 1210 (e.g., assembled into the garment 1210 or removably attached to the garment 1210, for example, using hook-and-look fasteners). In some implementations, the sensing electrodes 1212 can be permanently integrated into the garment 1210. The medical device controller 1220 can also be operatively coupled to the therapy electrodes 1214. For example, the therapy electrodes 1214 can also be assembled into the garment 1210, or, in some implementations, the therapy electrodes 1214 can be permanently integrated into the garment 1210. As shown in FIG. 12, the sensing electrodes 1212 and/or the therapy electrodes 1214 can be directly operatively coupled to the medical device controller 1220 and/or operatively coupled to the medical device controller 1220 through the connection pod 1230. Component configurations other than those shown in FIG. 12 are also possible. For example, the sensing electrodes 1212 can be configured to be attached at various positions about the body of the patient 1202. In some implementations, the sensing electrodes 1212 and/or at least one of the therapy electrodes 1214 can be included on a single integrated patch and adhesively applied to the patient's body. In some implementations, the sensing electrodes 1212 and/or at least one of the therapy electrodes 1214 can be included in multiple patches and adhesively applied to the patient's body.

Such patches may be in a wired (e.g., via the connection pod 1230) or wireless connection with the medical device controller 1220.

The sensing electrodes 1212 can be configured to detect one or more cardiac signals. Examples of such signals include ECG signals and/or sensed cardiac physiological signals from the patient 1202. In this way, in some implementations, the sensing electrodes 1212 may configured similarly to the ECG electrodes 114 discussed above. In some implementations, the sensing electrodes 1212 can include additional components such as accelerometers, acoustic signal detecting devices, and other measuring devices for recording additional parameters. For example, the sensing electrodes 1212 can also be configured to detect other types of patient physiological parameters and acoustic signals, such as tissue fluid levels, heart vibrations, lung vibrations, respiration vibrations, patient movement, etc. Example sensing electrodes 1212 include a metal electrode with an oxide coating such as tantalum pentoxide electrodes, as described in, for example, U.S. Pat. No. 6,253,099 entitled “Cardiac Monitoring Electrode Apparatus and Method,” the content of which is incorporated herein by reference.

In some examples, the therapy electrodes 1214 can also be configured to include sensors configured to detect ECG signals as well as, or in the alternative, other physiological signals from the patient 1202. The connection pod 1230 can, in some examples, include a signal processor configured to amplify, filter, and digitize these cardiac signals prior to transmitting the cardiac signals to the medical device controller 1220. One or more therapy electrodes 1214 can be configured to deliver one or more therapeutic defibrillating shocks to the body of the patient 1202 when the garment device 1200 determines that such treatment is warranted based on the signals detected by the sensing electrodes 1212 and processed by the medical device controller 1220. Example therapy electrodes 1214 can include conductive metal electrodes such as stainless-steel electrodes that include, in certain implementations, one or more conductive gel deployment devices configured to deliver conductive gel between the metal electrode and the patient's skin prior to delivery of a therapeutic shock.

Additionally, in some implementations, the garment device 1200 may include an RF sensor 1260. The RF sensor 1260 may include at least one RF antenna and RF circuitry configured to transmit RF waves into the thoracic region of the patient and receive RF waves transmitted through the thoracic region and/or reflected or scattered by the thoracic region. In this way, the RF sensor 1260 may include circuitry similar to the RF circuit shown in FIG. 11. In some examples, as shown in FIG. 12, the RF sensor 1260 may be mounted or otherwise incorporated into a strap 1262 over the patient's chest. The strap 1262 may be an integrated part of the garment 1210, or the strap 1262 may be connectable to the garment 1210, such as through mating hooks, snaps, hook-and-loop fabric, and/or the like.

In some implementations, a garment device 1200 as described herein can be configured to switch between a therapeutic device and a monitoring device that is configured to only monitor a patient (e.g., not provide or perform any therapeutic functions). For example, in such implementations, therapeutic components such as the therapy electrodes 1214 and associated circuitry may be decoupled from (or coupled to) or switch out of (or switched into) the garment device 1200. As an illustration, a garment device 1200 can have optional therapeutic elements (e.g., defibrillation and/or pacing electrode components and associated circuitry) that are configured to operate in a therapeutic mode. The optional therapeutic elements may be physically decoupled from the garment device 1200 as a means to convert the therapeutic garment device into a monitoring garment device for a specific use (e.g., for operating in a monitoring-only mode) for a patient. Alternatively, the optional therapeutic elements may be deactivated (e.g., by means of a physical or software switch), essentially rendering the therapeutic garment device as a monitoring garment device for a specific physiological purpose for a particular patient. As an example of a software switch, an authorized person may be able to access a protected user interface of the garment device 1200 and select a preconfigured option or perform some other user action via the user interface to deactivate the therapeutic elements of the garment device 1200.

FIG. 13 illustrates a sample component-level view of the medical device controller 1220. As shown in FIG. 13, the medical device controller 1220 can include a therapy delivery circuit 1302, a data storage 1304, a network interface 1306, a user interface 1308, at least one battery 1310, a sensor interface 1312, an alarm manager 1314, and at least one processor 1318. As described above, in some implementations, the garment device 1200 may not deliver therapy and instead may be used only for monitoring the patient 1202. As such, a monitoring garment device 1200 may include a medical device controller 1220 that includes like components as those described above but does not include a therapy delivery circuit 1302 (as shown in dotted lines).

The therapy delivery circuit 1302 can be coupled to the therapy electrodes 1214 configured to provide therapy to the patient 1202. For example, the therapy delivery circuit 1302 can include, or be operably connected to, circuitry components that are configured to generate and provide a therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit 1302 and under the control of one or more processors (e.g., processor 1318) to provide, for example, one or more pacing, defibrillation, or cardioversion therapeutic pulses. For example, acing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

The capacitors can include a parallel-connected capacitor bank consisting of a plurality of capacitors (e.g., two, three, four or more capacitors). These capacitors can be switched into a series connection during discharge for a defibrillation pulse. For example, four capacitors of approximately 650 μF can be used. The capacitors can have, for example, between 350 to 500 V surge rating and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 1302 can be configured to perform the switching and pulse delivery operations, e.g., under control of the processor 1318. As the energy is delivered to the patient 1202, the amount of energy being delivered can be tracked. For example, the amount of energy can be kept to a predetermined constant value even as the pulse waveform is dynamically controlled based on factors, such as the patient's body impedance, while the pulse is being delivered.

The data storage 1304 can include one or more of non-transitory computer readable media, such as flash memory, solid state memory, magnetic memory, optical memory, cache memory, combinations thereof, and others. The data storage 1304 can be configured to store executable instructions and data used for operation of the medical device controller 1220. In some implementations, the data storage 1304 can include sequences of executable instructions that, when executed, are configured to cause the processor 1318 to perform one or more functions.

In some examples, the network interface 1306 can facilitate the communication of information between the medical device controller 1220 and one or more devices or entities over a communications network. For example, the network interface 1306 can be configured to communicate with the remote server 102 or other similar computing device. The network interface 1306 can include communications circuitry for transmitting data in accordance with a Bluetooth® wireless standard for exchanging such data over short distances to an intermediary device(s) (e.g., the portable gateway 108 or another base station, “hotspot” device, smartphone, tablet, portable computing device, and/or other device in proximity with the garment device 1200). The intermediary device(s) may in turn communicate the data to the remote server 102 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 102 over a Wi-Fi communications link based on the IEEE 802.11 standard. In some implementations, the network interface 1306 may be configured to instead communicate directly with the remote server 102 without the use of intermediary device(s). In such implementations, the network interface 1306 may use any of the communications links and/or protocols provided above.

In some implementations, the user interface 1308 may include one or more physical interface devices, such as input device, output devices, and combination input/output devices, and a software stack configured to drive operation of the devices. These user interface elements may render visual, audio, and/or tactile content. Thus, the user interface 1308 may receive inputs and/or provide outputs, thereby enabling a user to interact with the medical device controller 1220.

The medical device controller 1220 can also include at least one battery 1310 configured to provide power to one or more components integrated in the medical device controller 1220. The battery 1310 can include a rechargeable multi-cell battery pack. In one example implementation, the battery 1310 can include three or more cells (e.g., 2200 mA lithium ion cells) that provide electrical power to the other device components within the medical device controller 1220. For example, the battery 1310 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 1220.

The sensor interface 1312 can be coupled to one or more sensors configured to monitor one or more physiological parameters of the patient. As shown, the sensors may be coupled to the medical device controller 1220 via a wired or wireless connection. The sensors can include one or more sensing electrodes 1212 (e.g., ECG electrodes). In some embodiments, as further shown in FIG. 13, the sensors may include additional sensors, such as heart vibrations sensor 1324 (not shown in FIG. 12) and tissue fluid monitor 1326 (e.g., based on ultra-wide band radiofrequency devices). The tissue fluid monitor 1326 may be, for example, the RF sensor 1260 shown in FIG. 12. The sensor interface 1312 can be coupled to any one or combination of sensing electrodes/other sensors to receive other patient data indicative of patient parameters. Once data from the sensors has been received by the sensor interface 1312, the data can be directed by the processor 1318 to an appropriate component within the medical device controller 1220. For example, if heart data is collected by the heart vibrations sensor 1324 and transmitted to the sensor interface 1312, the sensor interface 1312 can transmit the data to the processor 1318, which, in turn, relays the data to a cardiac event detector (e.g., implemented by the processor 1318 executing sequences of instructions stored in the data storage 1304). The cardiac event data can also be stored in the data storage 1304.

In some implementations, the alarm manager 1314 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 102 or may be implemented as one or more separate systems). For example, when the processor 1318 determines using data from the sensing electrodes 1212 that the patient is experiencing a treatable arrhythmia, the alarm manager 1314 may issue an alarm via the user interface 1308 that the patient is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient that the patient can abort the delivery of the defibrillating shock by interacting with the user interface 1308. For instance, the patient may be able to press a user response button or user response buttons on the user interface 1308, after which the alarm manager 1314 will cease issuing an alert and the medical device controller 1220 will no longer prepare to deliver the defibrillating shock.

The alarm manager 1314 can be implemented using hardware or a combination of hardware and software. For instance, in some examples, the alarm manager 1314 can be implemented as a software component that is stored within the data storage 1304 and executed by the processor 1318. In this example, the instructions included in the alarm manager 1314 can cause the processor 1318 to configure alarm profiles and notify intended recipients using the alarm profiles. In other examples, the alarm manager 1314 can be an application-specific integrated circuit (ASIC) that is coupled to the processor 1318 and configured to manage alarm profiles and notify intended recipients using alarms specified within the alarm profiles. Thus, examples of the alarm manager 1314 are not limited to a particular hardware or software implementation.

In some implementations, the processor 1318 includes one or more processors (or one or more processor cores) that each are configured to perform a series of instructions that result in the manipulation of data and/or the control of the operation of the other components of the medical device controller 1220. In some implementations, when executing a specific process (e.g., cardiac monitoring), the processor 1318 can be configured to make specific logic-based determinations based on input data received. The processor 1318 may be further configured to provide one or more outputs that can be used to control or otherwise inform subsequent processing to be carried out by the processor 1318 and/or other processors or circuitry with which the processor 1318 is communicably coupled. Thus, the processor 1318 reacts to a specific input stimulus in a specific way and generates a corresponding output based on that input stimulus. In some example cases, the processor 1318 can proceed through a sequence of logical transitions in which various internal register states and/or other bit cell states internal or external to the processor 1318 may be set to logic high or logic low.

As referred to herein, the processor 1318 can be configured to execute a function where software is stored in a data store (e.g., the data storage 1304) coupled to the processor 1318, the software being configured to cause the processor 1318 to proceed through a sequence of various logic decisions that result in the function being executed. The various components that are described herein as being executable by the processor 1318 can be implemented in various forms of specialized hardware, software, or a combination thereof. For example, the processor 1318 can be a DSP such as a 24-bit DSP processor. As another example, the processor 1318 can be a multi-core processor, e.g., having two or more processing cores. As another example, the processor 1318 can be an ARM processor, such as a 32-bit ARM processor. The processor 1318 can execute an embedded operating system and further execute services provided by the operating system, where these services can be used for file system manipulation, display and audio generation, basic networking, firewalling, data encryption, communications, and/or the like.

The embodiments of an ambulatory medical device 100 shown in FIGS. 1-3 and 6-13 and described above are examples, and other embodiments of an ambulatory medical device 100 may be contemplated herein. Additionally features of the ambulatory medical device 100 shown in FIGS. 1-3 and 6-13 may be altered, combined, or switched out, in some implementations.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A patient monitoring system for thoracic fluid measurement from a patient, comprising:
an ambulatory medical device configured to be worn by the patient for an extended period of time, comprising:
at least one radiofrequency (RF) antenna and associated RF circuitry configured to transmit RF waves in a range from about 0.1 GHZ to about 5 GHZ towards a thoracic region of the patient and receive RF waves reflected from the thoracic region and/or transmitted through the thoracic region to generate at least one RF value therefrom; and
a remote service in communication with the ambulatory medical device, the remote server comprising:
a memory implemented in a non-transitory media; and;
a processor in communication with the memory, the processor configured to:
receive at least one RF value for the patient,
retrieve at least one weighting parameter from the memory, the at least one weighting parameter determined by a computer-tomography (CT) process comprising:
receiving reference thoracic fluid information for a patient population;
producing RF-based thoracic fluid information for the patient population; and
deriving the at least one weighting parameter by optimizing an association of the RF-based thoracic fluid information with the reference CT scan-based thoracic fluid information for the patient population;
apply the at least one weighting parameter to the at least one RF value to determine an amount of thoracic fluid in the patient, and
output the determined amount of thoracic fluid in the patient.

2-6. (canceled)

7. The patient monitoring system of claim 1, wherein the ambulatory medical device further comprises a plurality of ECG electrodes configured to sense ECG signals of the patient.

8. The patient monitoring system of claim 1, wherein the ambulatory medical device further comprises a motion sensor configured to acquire motion signals associated with the patient.

9. The patient monitoring system of claim 1, wherein the ambulatory medical device further comprises a garment configured to be worn about a torso of the patient.

10. The patient monitoring system of claim 9, wherein the ambulatory medical device further comprises a plurality of therapy electrodes configured to deliver a therapeutic shock to the patient.

11. The patient monitoring system of claim 1, wherein the ambulatory medical device further comprises an adhesive patch configured to be adhered to skin of the patient and a monitoring unit configured to be removably attached to the adhesive patch, wherein the monitoring unit comprises the at least one RF antenna and associated RF circuitry.

12. (canceled)

13. The patient monitoring system of claim 1, wherein the CT process further comprises.
identifying patient health information for the patient population; and
deriving the at least one weighting parameter by further optimizing an association of the RF-based thoracic fluid information and the patient health information with the reference CT scan-based thoracic fluid information for the patient population.

14. The patient monitoring system of claim 13, wherein the processor is further configured to
identify at least one patient health value for the patient;
wherein applying the at least one weighting parameter comprises applying the at least one weighting parameter to the at least one RF value and to the at least one patient health value to determine the amount of thoracic fluid in the patient.

15. The patient monitoring system of claim 14, wherein the patient health information for the patient population comprises demographic information for the patient population, and wherein the at least one patient health value for the patient comprises at least one demographic of the patient.

16. The patient monitoring system of claim 14, wherein the patient health information for the patient population comprises biometric information for the patient population, and wherein the at least one patient health value for the patient comprises at least one biometric of the patient.

17. The patient monitoring system of claim 14, wherein the patient health information for the patient population comprises anthropomorphic information for the patient population, and wherein the at least one patient health value for the patient comprises at least one anthropomorphic parameter of the patient.

18. The patient monitoring system of claim 14, wherein the patient health information for the patient population comprises at least one of gender information, age information, height information, weight information, body mass index (BMI) information, heart rate information, respiration rate information, heart failure type information, comorbidity information, cardiac events information, chest circumference information, or thoracic thickness information for the patient population; and
wherein the at least one patient health value for the patient comprises at least one of gender, age, height, weight, BMI, a heart rate, a respiration rate, a heart failure type, a comorbidity, a cardiac event, a chest circumference, or a thoracic tissue thickness of the patient.

19. The patient monitoring system of claim 14, wherein identifying the at least one patient health value for the patient comprises receiving the at least one patient health value as a user input.

20-21. (canceled)

22. The patient monitoring system of claim 1, wherein producing the RF-based thoracic fluid information for the patient population comprises:
receiving RF value information for the patient population; and
for each individual of the patient population, indexing the individual's RF value information by normalizing the RF value information to a baseline.

23. The patient monitoring system of claim 22, wherein applying the at least one weighting parameter to the at least one RF value comprises indexing the at least one RF value by normalizing the at least one RF value to the baseline.

24. The patient monitoring system of claim 31, wherein producing the RF-based thoracic fluid information for the patient population comprises:
receiving RF value information for the patient population; and
for each individual of the patient population, transforming the individual's RF value information using an alpha function, wherein the alpha function comprises an exponential function based on a rate of attenuation in the received RF waves reflected from the thoracic region and/or transmitted through the thoracic region compared to the RF waves transmitted from the at least one RF antenna.

25. The patient monitoring system of claim 24, wherein applying the at least one weighting parameter to the at least one RF value comprises transforming the at least one RF value using the alpha function.

26-84. (canceled)

85. The patient monitoring system of claim 14, wherein the ambulatory medical device further comprises an adhesive patch configured to be adhered to skin of the patient and a monitoring unit configured to be removably attached to the adhesive patch, wherein the monitoring unit comprises the at least one RF antenna and associated RF circuitry.

86. The patient monitoring system of claim 22, wherein the ambulatory medical device further comprises an adhesive patch configured to be adhered to skin of the patient and a monitoring unit configured to be removably attached to the adhesive patch, wherein the monitoring unit comprises the at least one RF antenna and associated RF circuitry.

87. The patient monitoring system of claim 24, wherein the ambulatory medical device further comprises an adhesive patch configured to be adhered to skin of the patient and a monitoring unit configured to be removably attached to the adhesive patch, wherein the monitoring unit comprises the at least one RF antenna and associated RF circuitry.
